SEQUENCE LISTING (21 sequences)

SEQ ID NO: 1 - protein, 418 aa, Artificial Sequence
  sequence for VioA
MKHSSDICIVGAGISGLTCASHLLDSPACRGLSLRIFDMQQEAGGRIRSKMLDGKASIELGAGRYSPQLHPHFQSAMQHYSQKSEVYPFTQLKFKSHVQQKLKRAMNELSPRLKEHGKESFLQFVSRYQGHDSAVGMIRSMGYDALFLPDISAEMAYDIVGKHPEIQSVTDNDANQWFAAETGFAGLIQGIKAKVKAAGARFSLGYRLLSVRTDGDGYLLQLAGDDGWKLEHRTRHLILAIPPSAMAGLNVDFPEAWSGARYGSLPLFKGFLTYGEPWWLDYKLDDQVLIVDNPLRKIYFKGDKYLFFYTDSEMANYWRGCVAEGEDGYLEQIRTHLASALGIVRERIPQPLAHVHKYWAHGVEFCRDSDIDHPSALSHRDSGIIACSDAYTEHCGWMEGGLLSAREASRLLLQRIAA

SEQ ID NO: 2 - protein, 998 aa, Artificial Sequence
  sequence for VioB
MSILDFPRIHFRGWARVNAPTANRDPHGHIDMASNTVAMAGEPFDLARHPTEFHRHLRSLGPRFGLDGRADPEGPFSLAEGYNAAGNNHFSWESATVSHVQWDGGEADRGDGLVGARLALWGHYNDYLRTTFNRARWVDSDPTRRDAAQIYAGQFTISPAGAGPGTPWLFTADIDDSHGARWTRGGHIAERGGHFLDEEFGLARLFQFSVPKDHPHFLFHPGPFDSEAWRRLQLALEDDDVLGLTVQYALFNMSTPPQPNSPVFHDMVGVVGLWRRGELASYPAGRLLRPRQPGLGDLTLRVNGGRVALNLACAIPFSTRAAQPSAPDRLTPDLGAKLPLGDLLLRDEDGALLARVPQALYQDYWTNHGIVDLPLLREPRGSLTLSSELAEWREQDWVTQSDASNLYLEAPDRRHGRFFPESIALRSYFRGEARARPDIPHRIEGMGLVGVESRQDGDAAEWRLTGLRPGPARIVLDDGAEAIPLRVLPDDWALDDATVEEVDYAFLYRHVMAYYELVYPFMSDKVFSLADRCKCETYARLMWQMCDPQNRNKSYYMPSTRELSAPKARLFLKYLAHVEGQARLQAPPPAGPARIESKAQLAAELRKAVDLELSVMLQYLYAAYSIPNYAQGQQRVRDGAWTAEQLQLACGSGDRRRDGGIRAALLEIAHEEMIHYLVVNNLLMALGEPFYAGVPLMGEAARQAFGLDTEFALEPFSESTLARFVRLEWPHFIPAPGKSIADCYAAIRQAFLDLPDLFGGEAGKRGGEHHLFLNELTNRAHPGYQLEVFDRDSALFGIAFVTDQGEGGALDSPHYEHSHFQRLREMSARIMAQSAPFEPALPALRNPVLDESPGCQRVADGRARALMALYQGVYELMFAMMAQHFAVKPLGSLRRSRLMNAAIDLMTGLLRPLSCALMNLPSGIAGRTAGPPLPGPVDTRSYDDYALGCRMLARRCERLLEQASMLEPGWLPDAQMELLDFYRRQMLDLACGKLSREA

SEQ ID NO: 3 - protein, 429 aa, Artificial Sequence
  sequence for VioC
MKRAIIVGGGLAGGLTAIYLAKRGYEVHVVEKRGDPLRDLSSYVDVVSSRAIGVSMTVRGIKSVLAAGIPRAELDACGEPIVAMAFSVGGQYRMRELKPLEDFRPLSLNRAAFQKLLNKYANLAGVRYYFEHKCLDVDLDGKSVLIQGKDGQPQRLQGDMIIGADGAHSAVRQAMQSGLRRFEFQQTFFRHGYKTLVLPDAQALGYRKDTLYFFGMDSGGLFAGRAATIPDGSVSIAVCLPYSGSPSLTTTDEPTMRAFFDRYFGGLPRDARDEMLRQFLAKPSNDLINVRSSTFHYKGNVLLLGDAAHATAPFLGQGMNMALEDARTFVELLDRHQGDQDKAFPEFTELRKVQADAMQDMARANYDVLSCSNPIFFMRARYTRYMHSKFPGLYPPDMAEKLYFTSEPYDRLQQIQRKQNVWYKIGRVN

SEQ ID NO: 4 - protein, 373 aa, Artificial Sequence
  sequence for VioD
MKILVIGAGPAGLVFASQLKQARPLWAIDIVEKNDEQEVLGWGVVLPGRPGQHPANPLSYLDAPERLNPQFLEDFKLVHHNEPSLMSTGVLLCGVERRGLVHALRDKCRSQGIAIRFESPLLEHGELPLADYDLVVLANGVNHKTAHFTEALVPQVDYGRNKYIWYGTSQLFDQMNLVFRTHGKDIFIAHAYKYSDTMSTFIVECSEETYARARLGEMSEEASAEYVAKVFQAELGGHGLVSQPGLGWRNFMTLSHDRCHDGKLVLLGDALQSGHFSIGHGTTMAVVVAQLLVKALCTEDGVPAALKRFEERALPLVQLFRGHADNSRVWFETVEERMHLSSAEFVQSFDARRKSLPPMPEALAQNLRYALQR

SEQ ID NO: 5 - protein, 191 aa, Artificial Sequence
  sequence for VioE
MENREPPLLPARWSSAYVSYWSPMLPDDQLTSGYCWFDYERDICRIDGLFNPWSERDTGYRLWMSEVGNAASGRTWKQKVAYGRERTALGEQLCERPLDDETGPFAELFLPRDVLRRLGARHIGRRVVLGREADGWRYQRPGKGPSTLYLDAASGTPLRMVTGDEASRASLRDFPNVSEAEIPDAVFAAKR

SEQ ID NO: 6 - protein, 2489 aa, Artificial Sequence
  violacein single operon reading frame comprising the VioA, VioB, VioC, VioD and VioE polypeptides
MKHSSDICIVGAGISGLTCASHLLDSPACRGLSLRIFDMQQEAGGRIRSKMLDGKASIELGAGRYSPQLHPHFQSAMQHYSQKSEVYPFTQLKFKSHVQQKLKRAMNELSPRLKEHGKESFLQFVSRYQGHDSAVGMIRSMGYDALFLPDISAEMAYDIVGKHPEIQSVTDNDANQWFAAETGFAGLIQGIKAKVKAAGARFSLGYRLLSVRTDGDGYLLQLAGDDGWKLEHRTRHLILAIPPSAMAGLNVDFPEAWSGARYGSLPLFKGFLTYGEPWWLDYKLDDQVLIVDNPLRKIYFKGDKYLFFYTDSEMANYWRGCVAEGEDGYLEQIRTHLASALGIVRERIPQPLAHVHKYWAHGVEFCRDSDIDHPSALSHRDSGIIACSDAYTEHCGWMEGGLLSAREASRLLLQRIAARAEGRGSLLTCGDVEENPGPMSILDFPRIHFRGWARVNAPTANRDPHGHIDMASNTVAMAGEPFDLARHPTEFHRHLRSLGPRFGLDGRADPEGPFSLAEGYNAAGNNHFSWESATVSHVQWDGGEADRGDGLVGARLALWGHYNDYLRTTFNRARWVDSDPTRRDAAQIYAGQFTISPAGAGPGTPWLFTADIDDSHGARWTRGGHIAERGGHFLDEEFGLARLFQFSVPKDHPHFLFHPGPFDSEAWRRLQLALEDDDVLGLTVQYALFNMSTPPQPNSPVFHDMVGVVGLWRRGELASYPAGRLLRPRQPGLGDLTLRVNGGRVALNLACAIPFSTRAAQPSAPDRLTPDLGAKLPLGDLLLRDEDGALLARVPQALYQDYWTNHGIVDLPLLREPRGSLTLSSELAEWREQDWVTQSDASNLYLEAPDRRHGRFFPESIALRSYFRGEARARPDIPHRIEGMGLVGVESRQDGDAAEWRLTGLRPGPARIVLDDGAEAIPLRVLPDDWALDDATVEEVDYAFLYRHVMAYYELVYPFMSDKVFSLADRCKCETYARLMWQMCDPQNRNKSYYMPSTRELSAPKARLFLKYLAHVEGQARLQAPPPAGPARIESKAQLAAELRKAVDLELSVMLQYLYAAYSIPNYAQGQQRVRDGAWTAEQLQLACGSGDRRRDGGIRAALLEIAHEEMIHYLVVNNLLMALGEPFYAGVPLMGEAARQAFGLDTEFALEPFSESTLARFVRLEWPHFIPAPGKSIADCYAAIRQAFLDLPDLFGGEAGKRGGEHHLFLNELTNRAHPGYQLEVFDRDSALFGIAFVTDQGEGGALDSPHYEHSHFQRLREMSARIMAQSAPFEPALPALRNPVLDESPGCQRVADGRARALMALYQGVYELMFAMMAQHFAVKPLGSLRRSRLMNAAIDLMTGLLRPLSCALMNLPSGIAGRTAGPPLPGPVDTRSYDDYALGCRMLARRCERLLEQASMLEPGWLPDAQMELLDFYRRQMLDLACGKLSREAQCTNYALLKLAGDVESNPGPMKRAIIVGGGLAGGLTAIYLAKRGYEVHVVEKRGDPLRDLSSYVDVVSSRAIGVSMTVRGIKSVLAAGIPRAELDACGEPIVAMAFSVGGQYRMRELKPLEDFRPLSLNRAAFQKLLNKYANLAGVRYYFEHKCLDVDLDGKSVLIQGKDGQPQRLQGDMIIGADGAHSAVRQAMQSGLRRFEFQQTFFRHGYKTLVLPDAQALGYRKDTLYFFGMDSGGLFAGRAATIPDGSVSIAVCLPYSGSPSLTTTDEPTMRAFFDRYFGGLPRDARDEMLRQFLAKPSNDLINVRSSTFHYKGNVLLLGDAAHATAPFLGQGMNMALEDARTFVELLDRHQGDQDKAFPEFTELRKVQADAMQDMARANYDVLSCSNPIFFMRARYTRYMHSKFPGLYPPDMAEKLYFTSEPYDRLQQIQRKQNVWYKIGRVNRAEGRGSLLTCGDVEENPGPMKILVIGAGPAGLVFASQLKQARPLWAIDIVEKNDEQEVLGWGVVLPGRPGQHPANPLSYLDAPERLNPQFLEDFKLVHHNEPSLMSTGVLLCGVERRGLVHALRDKCRSQGIAIRFESPLLEHGELPLADYDLVVLANGVNHKTAHFTEALVPQVDYGRNKYIWYGTSQLFDQMNLVFRTHGKDIFIAHAYKYSDTMSTFIVECSEETYARARLGEMSEEASAEYVAKVFQAELGGHGLVSQPGLGWRNFMTLSHDRCHDGKLVLLGDALQSGHFSIGHGTTMAVVVAQLLVKALCTEDGVPAALKRFEERALPLVQLFRGHADNSRVWFETVEERMHLSSAEFVQSFDARRKSLPPMPEALAQNLRYALQRRAEGRGSLLTCGDVEENPGPMENREPPLLPARWSSAYVSYWSPMLPDDQLTSGYCWFDYERDICRIDGLFNPWSERDTGYRLWMSEVGNAASGRTWKQKVAYGRERTALGEQLCERPLDDETGPFAELFLPRDVLRRLGARHIGRRVVLGREADGWRYQRPGKGPSTLYLDAASGTPLRMVTGDEASRASLRDFPNVSEAEIPDAVFAAKR

SEQ ID NO: 7 - DNA, 7467 bp, Artificial Sequence
  violacein ORF DNA sequence
atgaaacactcttctgatatttgtatagttggggcagggatatcaggcctcacctgtgcttcacaccttcttgatagcccagcttgcaggggcctgtcacttcgaatttttgacatgcaacaggaggccggcggacggatccgctctaagatgcttgatggcaaggcgtctatcgaactcggcgccggacggtactctccgcaacttcacccccacttccaaagtgcaatgcaacactacagtcaaaaatccgaggtctacccattcacccaattgaagttcaaatcccatgttcaacagaaactcaaacgggccatgaacgaactgtcaccgcgccttaaggagcacggaaaggagagctttctccagtttgtgtctcgctaccagggtcatgactccgctgtagggatgattaggtccatggggtatgatgccctctttctcccggatatatcagctgaaatggcttatgacattgttggcaagcatcccgaaattcagtctgtcacggacaacgatgccaaccagtggtttgcagcagaaacaggctttgcgggccttatacagggaattaaagccaaagtaaaggccgctggtgctcgattctcacttggctatcgactcctcagtgttaggacagatggtgatggctatctcttgcaattggccggcgacgatggttggaagttggagcaccgaacccgccacttgatcctcgccatcccaccttctgcaatggctggacttaacgtcgacttccctgaagcttggtcaggggcacgatatggctcactccctctcttcaaagggttccttacttacggagagccttggtggcttgactataagcttgacgaccaggttctcattgtagataatccgctcaggaagatttatttcaaaggcgacaagtacctcttcttctatactgattctgagatggctaactattggaggggctgcgtagcggaaggggaggacgggtatctggaacaaatacgaacccacctggccagtgcccttggcatagtacgggagcggataccacagcctctcgctcatgtgcacaagtattgggcgcatggtgtcgaattctgccgcgactctgacatcgatcacccctccgccctgagtcacagggattcaggtattattgcttgcagcgatgcgtataccgaacattgcggttggatggaaggaggtctgctgtctgcccgagaagcctcccgactgctccttcagagaatcgcggcaagagcagaagggcgggggagccttcttacatgtggagacgtggaggaaaatccaggacctatgtcaattctggattttccgcgcatccattttagaggctgggcgagagtcaacgctccaacagccaaccgggacccgcatggccacatcgatatggcgtctaacacagtggcaatggcaggggagccattcgatcttgctagacacccgacagagttccatcgacatttgcgaagtttgggaccgcggttcggcctcgacgggagagcagacccggaaggtccgttctctcttgcggaggggtataatgccgcaggcaacaatcacttttcttgggaatctgctacggtatcccatgtgcaatgggatgggggtgaagcagaccgaggtgatgggcttgtcggcgcaagactcgcactgtggggacactataacgattacttgcgcaccaccttcaaccgagcgcgatgggtcgacagcgatccgacccggcgggatgccgctcagatatatgctgggcaatttaccatttccccagccggggccgggccagggacgccatggttgttcacggcagacattgatgactcccatggcgcccggtggacccgaggaggtcacatcgcggaaagggggggtcattttttggacgaggaatttggcctggcaagactttttcaattctccgttccgaaagaccacccacattttcttttccatcctggacctttcgattccgaagcttggagaaggctgcaactggcgttggaggacgacgatgtactgggcctgactgtccagtacgctctttttaacatgagtactccaccacaacccaacagcccagtcttccacgatatggtaggagtggttgggttgtggagaagaggagagctcgcaagctatcccgcgggacgactgcttcgcccccgacagccggggctcggagatcttacgcttagagtcaacggcggcagagttgctcttaacctcgcatgcgcaattccattctctactcgggcagctcagccctccgctccggataggttgacacctgacctcggagcaaaactgccgctcggcgatcttctccttagggacgaggacggtgcgctgctggccagggtaccccaagcgctttaccaagattactggacgaaccatggaatagtggacttgcctctccttcgggaacctagaggctcacttacattgtcctccgagctggcagagtggagggaacaggactgggttacacaaagcgacgcgtccaatttgtatcttgaagctcctgaccggcgccatgggcgattttttccggaaagtatagcgctcaggagctatttcagaggtgaagcaagggcgcgaccggacattccccatcggattgaaggcatgggcctcgtgggggtcgagagccggcaggacggggatgccgcagaatggcgcttgacaggattgaggccgggtccggcaaggattgtgctggatgatggggccgaggcaattccattgcgagtactgcccgatgactgggctttggacgatgcgactgtcgaagaagtagattacgcgtttctttacaggcacgttatggcttactacgaactggtatacccatttatgagcgataaggtattctcactggccgaccgatgcaaatgcgagacgtacgcgcgcctgatgtggcaaatgtgtgatcctcagaatcgcaataaaagttactacatgccgagtacgcgcgagctcagcgcaccaaaggctcgcctgtttctgaagtacttggcccatgtggaagggcaggcgaggttgcaagctcccccaccagccgggcccgccagaatagaaagtaaagcccaattggccgcagagttgcgcaaagccgtcgatttggaactctccgtcatgcttcaatatctctacgcagcgtattctataccgaactacgcacagggtcaacaaagagtcagagacggtgcgtggaccgccgaacagcttcaacttgcatgcggtagcggtgataggcgaagggacggtggtatacgcgcggcattgttggaaattgcccacgaagaaatgatacattacctcgtggtcaacaatcttctcatggcgctgggcgaaccattctatgccggcgtgccccttatgggggaagcagctaggcaagctttcggcctggacacagaatttgctcttgagccgttttccgagtcaactttggcacgattcgtccggttggaatggccacactttatcccagccccaggaaagagtatagcggattgttatgctgcaatccgacaggcttttcttgatctccccgatctctttggcggtgaggccgggaaacgaggtggcgagcaccacctcttcttgaatgaattgaccaaccgcgcacacccgggttaccaactggaagtatttgatagggatagcgcgttgtttggaatagcgtttgtcaccgatcaaggtgaaggcggtgcactcgacagtccgcactatgaacactcccactttcagcggttgcgggaaatgagcgcacggataatggctcaatccgctcccttcgaacctgcccttccggccctcagaaaccccgttctcgatgagagcccaggctgccaacgggtggccgacgggcgcgcacgcgcgctgatggcactgtaccagggggtgtacgaactgatgttcgcaatgatggctcagcactttgctgtaaaaccgctcgggagtcttcgaaggtccaggttgatgaatgccgcaattgatttgatgaccgggctcctccgccctttgtcatgtgctctcatgaatttgccttcaggtatagcggggcgcaccgcaggaccgccacttccaggacccgttgacacgcgaagctacgacgattatgccctgggctgccgaatgctggcacgacgctgcgaacgactgcttgagcaagcgtccatgctggaacccggatggcttcccgacgcccagatggaactcctggatttctatcgacgccagatgctggatcttgcgtgcgggaagctgagtagggaggcgcagtgtactaactatgctctgttgaaattggctggggatgtcgaatccaatccaggccctatgaaacgagcaatcattgtcggcggcggcctcgccggtggcctgacagccatctatttggctaaacgcgggtatgaggtccatgtagtagagaagagaggtgatcctttgcgagatttgagcagctatgttgacgtggtatcttcccgggccatcggtgtcagtatgacggtcagaggcataaaatccgtgttggcggccggtatcccacgcgccgaactggatgcttgtggcgagccaattgtagcaatggcattctccgtaggcgggcaataccgaatgcgggaacttaaaccgctcgaggatttccggccactgtcattgaatcgggctgcgttccaaaaactgcttaataaatacgcaaaccttgcaggcgttaggtattatttcgagcacaagtgtctcgatgtcgatttggacgggaaaagtgttctgattcaaggaaaagacgggcaaccgcagcgccttcagggtgacatgataataggcgcggacggcgcgcacagcgccgtacgacaggccatgcaatctggactccggcggtttgaattccagcaaacatttttccgccatgggtataagactttggttctgcctgatgcgcaagctttggggtatcggaaagatacgctctatttctttgggatggatagtggagggcttttcgccggacgcgctgctacgattcccgacggaagtgtctcaatagcagtctgtcttccgtacagtggatccccgagccttacgactacggatgaaccgaccatgcgggcgtttttcgaccgctacttcggaggtttgccgagagatgctcgggacgaaatgctcaggcaattccttgccaaaccgagtaacgatttgatcaacgtgcggtcttccacatttcactataaaggtaacgtgctgttgctgggcgacgcagcccacgcaacagcaccgttcctggggcaagggatgaatatggcattggaagacgcgagaacgttcgtcgagttgcttgatcgccaccaaggtgatcaggataaagcgtttccggaatttacagagcttaggaaggttcaagccgatgctatgcaagacatggcacgagcgaactatgatgtgctcagctgtagtaacccgatcttttttatgagagcaagatatacgaggtacatgcatagtaaattcccaggtctgtacccccccgatatggctgagaaactctatttcacgtctgagccgtatgatcgattgcaacagatccagcgaaaacaaaatgtatggtataagattggtcgcgttaatcgagcagaagggcgagggtcactgttgacatgtggtgacgtggaagagaaccccggccctatgaagatcctcgtcatcggcgcgggaccagccggtttggtgtttgcgtcccaacttaaacaggcgaggcccctgtgggcgatagatatcgtcgaaaaaaacgatgaacaagaggtgcttggatggggggtggtcttgcctggtagaccgggtcagcaccctgcgaatccgcttagctacctcgacgcgcccgagaggctgaaccctcagttccttgaagacttcaaactggtgcatcataatgaaccaagtctcatgtctaccggagtacttttgtgcggggtcgagagacggggcctggtccatgctctgcgggataagtgcaggtcccaaggtatagctattaggtttgaaagtccattgcttgaacatggcgaacttcccttggcggattatgatcttgtggtactcgcaaacggagtgaaccataagaccgcgcattttaccgaggctctggttcctcaggtcgactatggtcgaaacaagtacatttggtacggcacctcccaacttttcgatcaaatgaacctggtatttaggacgcacggcaaagacattttcattgctcatgcgtataaatactccgacaccatgtccacgtttattgtcgagtgctctgaggagacgtacgctagggcccggctgggcgaaatgagtgaggaagcatcagcagaatacgtcgccaaggttttccaagcagaactcggagggcatgggctggtaagccaacccggattgggatggaggaacttcatgactcttagccacgatcgctgccatgacggaaaactcgtgttgttgggggacgcactccagagcggtcactttagtattggacacggtaccacgatggctgttgtggtagcacagttgcttgtcaaagcgttgtgcacagaggatggtgtacccgcagcgcttaagcgcttcgaggagagggctctgcccctggttcaacttttccgcggtcatgcggacaacagccgggtatggtttgaaacagttgaggagcgaatgcacttgtcctccgctgaatttgtccaaagctttgatgcccgccggaaaagtcttccgcctatgcctgaagcgcttgctcagaatcttcgatatgccctccagaggagggccgaggggcggggctcacttcttacgtgcggtgacgtagaagaaaatcccgggcctatggaaaaccgggaacctcccttgttgccagcacggtggtcctccgcatatgtctcctactggtcaccgatgttgccagacgatcagctgacctcagggtactgttggtttgattatgagagagacatctgcagaattgacggtctttttaacccctggtctgagagagataccggttacagactgtggatgtctgaagtagggaatgcagcgagtggtaggacctggaagcaaaaagtggcatacggcagggagcgaacggctttgggagaacagctttgcgagcgaccattggatgacgaaacaggcccctttgccgagttgttcctgccacgagacgtattgcgcagacttggagcacgacatataggacgccgggtagttctgggcagggaagccgatggatggagatatcagcgaccaggaaaagggccaagtaccctgtatctggatgcagccagcgggaccccacttcggatggtcactggagacgaagcgagtcgcgcttccttgagggattttcccaacgtttccgaagcggagataccggatgctgtttttgccgccaagcgc

SEQ ID NO: 8 - protein, 594 aa, Valeriana officinalis
  misc_feature (228)..(228): Xaa can be any naturally occurring amino acid
MITSSSSVRSLCCPKTSIISGKLLPSLLLTNVINVSNGTSSRACVSMSSLPVSKSTASSIAAPLVRDNGSALNFFPQAPQVEIDESSRIMELVEATRRTLRNESSDSTEKMRLIDSLQRLGLNHHFEQDIKEMLQDFANEHKNTNQDLFTTSLRFRLLRHNGFNVTPDVFNKFTEENGKFKESLGEDTIGILSLYEASYLGGKGEEILSEAMKFSESKLRESSGHVAXHIRRQIFQSLELPRHLRMARLESRRYIEEDYSNEIGADSSLLELAKLDFNSVQALHQMELTEISRWWKQLGLSDKLPFARDRPLECFLWTVGLLPEPKYSGCRIELAKTIAVLLVIDDIFDTYGSYDQLILFTNAIRRWDLDAMDELPEYMKICYMALYNTTNEICYKVLKENGWSVLPYLERTWIDMVEGFMLEAKWLNSGEQPNLEAYIENGVTTAGSYMALVHLFFLIGDGVNDENVKLLLDPYPKLFSSAGRILRLWDDLGTAKEEQERGDVSSSIQLYMKEKNVRSESEGREGIVEIIYNLWKDMNGELIGSNALPQAIIETSFNMARTSQVVYQHEDDTYFSSVDNYVQSLFFTPVSVSV

SEQ ID NO: 9 - protein, 718 aa, Aspergillus clavatus
MACKYSTLIDSSLYDREGLCPGIDLRRHVAGELEEVGAFRAQEDWRRLVGPLPKPYAGLLGPDFSFITGAVPECHPDRMEIVAYALEFGFMHDDVIDTDVNHASLDEVGHTLDQSRTGKIEDKGSDGKRQMVTQIIREMMAIDPERAMTVAKSWASGVRHSSRRKEDTNFKALEQYIPYRALDVGYMLWHGLVTFGCAITIPNEEEEEAKRLIIPALVQASLLNDLFSFEKEKNDANVQNAVLIVMNEHGCSEEEARDILKKRIRLECANYLRNVKETNARADVSDELKRYINVMQYTLSGNAAWSTNCPRYNGPTKFNELQLLRSEHGLAKYPSRWSQENRTSGLVEGDCHESKPNELKRKRNGVSVDDEMRTNGTNGAKKPAHVSQPSTDSIVLEDMVQLARTCDLPDLSDTVILQPYRYLTSLPSKGFRDQAIDSINKWLKVPPKSVKMIKDVVKMLHSASLMLDDLEDNSPLRRGKPSTHSIYGMAQTVNSATYQYITATDITAQLQNSETFHIFVEELQQLHVGQSYDLYWTHNTLCPTIAEYLKMVDMKTGGLFRMLTRMMIAESPVVDKVPNSDMNLFSCLIGRFFQIRDDYQNLASADYAKAKGFAEDLDEGKYSFTLIHCIQTLESKPELAGEMMQLRAFLMKRRHEGKLSQEAKQEVLVTMKKTESLQYTLSVLRELHSELEKEVENLEAKFGEENFTLRVMLELLKV

SEQ ID NO: 10 - protein, 486 aa, Panax ginseng
MAAAMVLFFSLSLLLLPLLLLFAYFSYTKRIPQKENDSKAPLPPGQTGWPLIGETLNYLSCVKSGVSENFVKYRKEKYSPKVFRTSLLGEPMAILCGPEGNKFLYSTEKKLVQVWFPSSVEKMFPRSHGESNADNFSKVRGKMMFLLKVDGMKKYVGLMDRVMKQFLETDWNRQQQINVHNTVKKYTVTMSCRVFMSIDDEEQVTRLGSSIQNIEAGLLAVPINIPGTAMNRAIKTVKLLTREVEAVIKQRKVDLLENKQASQPQDLLSHLLLTANQDGQFLSESDIASHLIGLMQGGYTTLNGTITFVLNYLAEFPDVYNQVLKEQVEIANSKHPKELLNWEDLRKMKYSWNVAQEVLRIIPPGVGTFREAITDFTYAGYLIPKGWKMHLIPHDTHKNPTYFPSPEKFDPTRFEGNGPAPYTFTPFGGGPRMCPGIEYARLVILIFMHNVVTNFRWEKLIPNEKILTDPIPRFAHGLPIHLHPHN

SEQ ID NO: 11 - protein, 457 aa, Panax ginseng
MEREMLSKTHIMFIPFPAQGHMSPMMQFAKRLAWKGLRITIVLPAQIRDFMQITNPLINTECISFDFDKDDGMPYSMQAYMGVVKLKVTNKLSDLLEKQRTNGYPVNLLVVDSLYPSRVEMCHQLGVKGAPFFTHSCAVGAIYYNARLGKLKIPPEEGLTSVSLPSIPLLGRDDLPIIRTGTFPDLFEHLGNQFSDLDKADWIFFNTFDKLENEEAKWLSSQWPITSIGPLIPSMYLDKQLPNDKDNGINFYKADVGSCIKWLDAKDPGSVVYASFGSVKHNLGDDYMDEVAWGLLHSKYHFIWVVIESERTKLSSDFLAEAEAEEKGLIVSWCPQLQVLSHKSIGSFMTHCGWNSTVEALSLGVPMVALPQQFDQPANAKYIVDVWQIGVRVPIGEEGVVLRGEVANCIKDVMEGEIGDELRGNALKWKGLAVEAMEKGGSSDKNIDEFISKLVSS

SEQ ID NO: 12 - protein, 711 aa, Arabidopsis thaliana
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 13 - protein, 769 aa, Panax ginseng
MWKQKGAQGNDPYLYSTNNFVGRQYWEFQPDAGTPEEREEVEKARKDYVNNKKLHGIHPCSDMLMRRQLIKESGIDLLSIPPLRLDENEQVNYDAVTTAVKKALRLNRAIQAHDGHWPAENAGSLLYTPPLIIALYISGTIDTILTKQHKKELIRFVYNHQNEDGGWGSYIEGHSTMIGSVLSYVMLRLLGEGLAESDDGNGAVERGRKWILDHGGAAGIPSWGKTYLAVLGVYEWEGCNPLPPEFWLFPSSFPFHPAKMWIYCRCTYMPMSYLYGKRYHGPITDLVLSLRQEIYNIPYEQIKWNQQRHNCCKEDLYYPHTLVQDLVWDGLHYFSEPFLKRWPFNKLRKRGLKRVVELMRYGATETRFITTGNGEKALQIMSWWAEDPNGDEFKHHLARIPDFLWIAEDGMTVQSFGSQLWDCILATQAIIATNMVEEYGDSLKKAHFFIKESQIKENPRGDFLKMCRQFTKGAWTFSDQDHGCVVSDCTAEALKCLLLLSQMPQDIVGEKPEVERLYEAVNVLLYLQSRVSGGFAVWEPPVPKPYLEMLNPSEIFADIVVEREHIECTASVIKGLMAFKCLHPGHRQKEIEDSVAKAIRYLERNQMPDGSWYGFWGICFLYGTFFTLSGFASAGRTYDNSEAVRKGVKFFLSTQNEEGGWGESLESCPSEKFTPLKGNRTNLVQTSWAMLGLMFGGQAERDPTPLHRAAKLLINAQMDNGDFPQQEITGVYCKNSMLHYAEYRNIFPLWALGEYRKRVWLPKHQQLKI

SEQ ID NO: 14 - DNA, 6 bp, Artificial Sequence
  consensus NFAT recognition sequence
ggaaaa

SEQ ID NO: 15 - protein, 112 aa, Artificial Sequence
  CD3-zeta endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 368 aa, Artificial Sequence
  4-1BB and CD3-zeta endodomains
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - protein, 152 aa, Artificial Sequence
  CD28 and CD3-zeta endodomains
SKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 188 aa, Artificial Sequence
  CD28, OX40 and CD3-zeta endodomains
SKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 4 aa, Artificial Sequence
  basic amino acid furin target sequence
  misc_feature (2)..(2): Xaa can be any naturally occurring amino acid
  MISC_FEATURE (3)..(3): Xaa may be Arg or Lys
RXXR

SEQ ID NO: 20 - protein, 7 aa, Artificial Sequence
  consensus Tobacco Etch Virus (TEV) cleavage site
ENLYFQS

SEQ ID NO: 21 - protein, 4 aa, Artificial Sequence
  ITAM (immunoreceptor tyrosine-based activation motif)
  misc_feature (2)..(3): Xaa can be any naturally occurring amino acid
  MISC_FEATURE (4)..(4): Xaa may be Leu or Ile
YXXX

(19) United States
(12) Patent Application Publication
(10) Pub. No.: US 2020/0345776 A1
(43) Pub. Date: Nov. 5, 2020

(54) CELL

(71) Applicant: AUTOLUS LIMITED, London (GB)

(72) Inventors: Simon Thomas, London (GB); Martin Pulé, London (GB); Paul Smith, London (GB); Isaac Gannon, London (GB); William Balland, London (GB)

(21) Appl. No.: 16/763,539

(22) PCT Filed: Nov. 12, 2018
(86) PCT No.: PCT/GB2018/053262

§ 371 Date: May 12, 2020

(30) Foreign Application Priority Data
Nov. 13, 2017 (GB) 1718697.4

(51) Int. Cl. A61K 35/17; C07K 14/725; A61P 35/00

(52) U.S. Cl. CPC A61K 35/17; A61P 35/00; C07K 14/7051

(57) ABSTRACT

The present invention relates to an engineered cell which comprises; (i) a chimeric antigen receptor (CAR) or a transgenic T-cell receptor (TCR); and (ii) one or more engineered polynucleotides which encode one or more enzymes which are capable of synthesising a therapeutic small molecule when expressed in combination in the cell.

### FIELD OF THE INVENTION

The present invention relates to an engineered cell which expresses a chimeric antigen receptor (CAR) or a transgenic T-cell receptor (TCR); and in particular to approaches to expand the therapeutic agents expressed by said cell.

### BACKGROUND TO THE INVENTION

Antigen-specific T-cells may be generated by selective expansion of peripheral blood T-cells natively specific for the target antigen. However, it is difficult and quite often impossible to select and expand large numbers of T-cells specific for most cancer antigens. Gene-therapy with integrating vectors affords a solution to this problem as transgenic expression of Chimeric Antigen Receptor (CAR) allows generation of large numbers of T-cells specific to any surface antigen by ex vivo viral vector transduction of a bulk population of peripheral blood T-cells.

CAR T-cells have been successful in lymphoid malignancies. However, additional challenges are presented when using CAR T-cell therapy to treat solid cancers. There are several reasons why lymphoid cancers may be more amenable to CAR T-cell therapy than solid cancers. By way of example, T-cells normally traffic to typical sites of disease of lymphoid tumours, but with solid tumours CAR T-cells must migrate to sites of disease. Hence, far fewer T-cells may gain access to a solid tumour.

Further, the solid tumour microenvironment can be hostile to T-cells. For instance, inhibitory receptors may be upregulated. The tumour microenvironment may contain diverse types of inhibitory cells such as inhibitory T-cells, myeloid or stromal cells. Hence, T-cells which gain access to the solid tumour may be inhibited in their activity. The factors noted above may also form a barrier which prevents the CAR T-cell from entering and engrafting in the solid tumour.

Further still, solid tumour cells may be more difficult to kill than lymphoid cancer cells. For example, lymphoid tumours are often close to apoptosis and a single CAR T-cell/tumour cell interaction may be sufficient to induce killing of the lymphoid tumour cells.

The tumour microenvironment may be modulated by concomitant administration of a systemic agent with CAR T-cells. The systemic agent might be an antibody that blocks an inhibitory pathway (e.g. PD1/PDL1); a small molecule which inhibits tumour metabolism (e.g. an IDO inhibitor) or a cytotoxic agent.

However, a limitation of such systemic approaches is that the systemic distribution of the agent may result in toxicity. Further, in some cases, the agent may be toxic to the CAR T-cell.

Alternatively, several strategies have been developed which involve engineering CAR T-cells to release protein factors which can alter the tumour microenvironment and increase access of T-cells and other immune cells into the tumour microenvironment.

These protein factors include cytokines, chemokines, scFv or antibodies which block inhibitory pathways or even enzymes which disrupt the integrity of the microenvironment.

Protein factors can easily be encoded within a CAR T-cell using an open-reading frame which encodes the factor to be co-expressed with the CAR. However, even when released into the tumour microenvironment by the CAR T-cells, proteins are limited in their biodistribution. By way of example, secreted proteins may not penetrate into cells and thus their activity may be limited to the modulation of surface receptors.

Accordingly, there remains a need for alternative approaches to improve the effectiveness of engineered cells, in particular engineered immune cells expressing a CAR or a transgenic TCR in targeting solid tumours.

### SUMMARY OF THE INVENTION

The present inventors now provide an engineered cell which encodes a transgenic synthetic biology pathway that enables the engineered cell to produce a small molecule, in particular a therapeutic small molecule. In contrast to proteins, small molecules can—for example—penetrate into cells and disrupt key intracellular pathways including signalling pathways and metabolic pathways.

Accordingly, in a first aspect the present invention provides an engineered cell which comprises; (i) a chimeric antigen receptor (CAR) or a transgenic T-cell receptor (TCR); and (ii) one or more engineered polynucleotides which encode one or more enzymes which are capable of synthesising a therapeutic small molecule when expressed in combination in the cell.

The one or more enzymes may be encoded by one or more engineered polynucleotides. The one or more enzymes may be encoded by one engineered polynucleotide. Suitably, the engineered polynucleotide may be an operon.

The one or more enzymes may be encoded in one or more open reading frames. The one or more enzymes may be encoded in a single open reading frame. Suitably, each enzyme may be separated by a cleavage site. The cleavage site may be a self-cleavage site, such as a sequence encoding a FMD-2A like peptide.

The one or more enzymes may comprise at least two, at least three, at least four or at least five, at least six, at least seven, at least eight, at least nine, at least ten, at least eleven, at least twelve, at least thirteen, at least fourteen, or at least fifteen enzymes.

The one or more enzymes may comprise at least two, at least three, at least four or at least five enzymes.

The therapeutic small molecule may be selected from a cytotoxic molecule; a cytostatic molecule; an agent which is capable of inducing differentiation of the tumour; and a proinflammatory molecule. Suitably, the therapeutic small molecule may be violacein or mycophenolic acid.

In one embodiment the therapeutic small molecule is violacein. The engineered polynucleotide may comprise one or more open reading frames encoding VioA, VioB, VioC, VioD and VioE enzymes required to synthesise violacein from tryptophan. Suitably, the engineered polynucleotide may comprise a single open reading frame encoding VioA, VioB, VioC, VioD and VioE enzymes required to synthesise violacein from tryptophan. The violacein operon may encode a polypeptide comprising the sequence shown as SEQ ID NO: 1 or a variant which has at least 80% sequence identity thereto.

In another embodiment, the small molecule is geraniol

The engineered cell may be further engineered to have reduced sensitivity to the therapeutic small molecule. For example, the therapeutic small molecule may be mycophenolic acid and the cell may further express a mutated inosine monophosphate dehydrogenase 2 which has reduced sensitivity to mycophenolate.

Suitably the expression of the one or more enzymes may be induced by the binding of an antigen to the CAR or transgenic TCR.

The expression of the one or more enzymes may be induced by a tumour microenvironment.

The expression of the one or more enzymes may be induced by the binding of a second small molecule to the cell. Suitably, the second small molecule may be a pharmaceutical small molecule.

The cell may be an alpha-beta T cell, a NK cell, a gamma-delta T cell or a cytokine-induced killer cell.

In a further aspect the present invention provides a nucleic acid construct which comprises: (i) a first nucleic acid sequence which encodes a chimeric antigen receptor (CAR) or a transgenic TCR; and (ii) one or more nucleic acid sequences which encode one or more enzymes which are capable of synthesising a therapeutic small molecule when expressed in combination in a cell.

Suitably, the one or more enzymes which are capable of synthesising a therapeutic small molecule when expressed in combination in a cell are encoded on a single nucleic acid sequence.

The first and second nucleic acid sequences may be separated by a co-expression site.

In a further aspect the present invention provides a kit of nucleic acid sequences comprising: (i) a first nucleic acid sequence which encodes a chimeric antigen receptor (CAR) or a transgenic TCR; and (ii) one or more nucleic acid sequences which encode one or more enzymes which are capable of synthesising a therapeutic small molecule when expressed in combination in a cell.

Suitably, the one or more enzymes which are capable of synthesising a therapeutic small molecule when expressed in combination in a cell are encoded on a single nucleic acid sequence.

In another aspect the present invention provides a vector which comprises a nucleic acid construct according to the present invention.

In another aspect the present invention provides a kit of vectors which comprises: (i) a first vector which comprises a nucleic acid sequence which encodes a chimeric antigen receptor (CAR) or a transgenic TCR; and (ii) one or more vector which encode one or more enzymes which are capable of synthesising a therapeutic small molecule when expressed in combination in a cell.

Suitably, the one or more enzymes which are capable of synthesising a therapeutic small molecule when expressed in combination in a cell are encoded by a single vector.

The nucleic acid construct, kit of nucleic acid sequences, vector or a kit of vectors according to the present invention may comprise one or more enzymes as defined for the first aspect of the present invention.

In a further aspect the present invention provides a pharmaceutical composition which comprises a cell; a nucleic acid construct; a first nucleic acid sequence and a second nucleic acid sequence; a vector; or a first and a second vector according to the present invention.

In a further aspect the present invention provides a pharmaceutical composition according to the present invention for use in treating and/or preventing a disease.

In another aspect the present invention relates to a method for treating and/or preventing a disease, which comprises the step of administering a pharmaceutical composition according to the present invention to a subject in need thereof.

The method may comprise the following steps:

(i) isolation of a cell containing sample;

(ii) transduction or transfection of the cell with a nucleic acid construct, a vector or a first and a second vector according to the present invention; and

(iii) administering the cells from (ii) to a subject.

The cell may be autologous. The cell may be allogenic.

In a further aspect the present invention relates to the use of a pharmaceutical composition according to present invention in the manufacture of a medicament for the treatment and/or prevention of a disease.

The disease may be cancer. The cancer may be a solid tumour cancer.

In another aspect the present invention relates to a method for making a cell according to the present invention which comprises the step of introducing: a nucleic acid construct; a first nucleic acid sequence and a second nucleic acid sequence; a vector or a first and a second vector of the present invention into the cell.

The cell may be from a sample isolated from a subject.

An advantage of the present invention is that it allows a very high local concentration of an otherwise toxic small molecule at the site of a solid tumour. The small molecule can easily diffuse from the engineered cell of the present invention and can diffuse into a tumour cell to enact a direct toxic or modulatory effect. Accordingly, production of a therapeutic small molecule by the engineered cell of the present invention can ameliorate some the difficulties associated with targeting a solid tumour whilst reducing the drawbacks of potentially toxic effects associated with systemic administration of the therapeutic small molecule.

### BRIEF DESCRIPTION OF THE FIGURES

FIG. 1—a) Schematic diagram illustrating a classical CAR. (b) to (d): Different generations and permutations of CAR endodomains: (b) initial designs transmitted ITAM signals alone through FcεR1-γ or CD3ζ endodomain, while later designs transmitted additional (c) one or (d) two co-stimulatory signals in the same compound endodomain.

FIG. 2—(a) Summary of the violacein biosynthetic pathway; (b) Operon for violacein converted into a eukaryotic format with all 5 enzymes coded for as a single frame separated by FMD-2A like peptides.

FIG. 3—Overview of the mevalonate pathway

FIG. 4—Overview of terpene biosynthesis

FIG. 5—Synthesis of ginsenosides from triterpene precursors

FIG. 6—Sensitivity of 4T1 or SKOV3 human cell lines to increasing geraniol concentrations

FIG. 7—Sensitivity of SKOV3 cells to the presence of geraniol producing CAR constructs

FIG. 8—Production of caffeine by a human cell line transduced with the caffeine biosynthetic genes CAXMT1 and CCS1 genes

FIG. 9—Caffeine expression in PBMCs isolated from 2 donors, in the presence of 100 μM xanthosine

FIG. 10—Toxicity of increasing violacein concentration on adherent tumour cell lines

FIG. 11—Production of violacein in SupT1 cells by dual transduction of SupT1 T cell line

FIG. 12—Violacein produced by SupT1 cells is toxic to SKOV3 tumour cells

### DETAILED DESCRIPTION OF THE INVENTION

One or More Enzymes

The present invention provides an engineered cell which comprises (i) a chimeric antigen receptor (CAR) or a transgenic T-cell receptor (TCR); and (ii) one or more engineered polynucleotides which encode one or more enzymes which are capable of synthesising a therapeutic small molecule when expressed in combination in the cell.

As used herein, an “engineered polynucleotide” refers to a polynucleotide which is not naturally present in the cell genome. Such engineered polynucleotides may be introduced into a cell using, for example, standard transduction or transfection methods as described herein. For example, engineered polynucleotide may be transferred to a cell using retroviral vectors.

A small molecule cannot be directly encoded by a simple gene in the manner by which a protein can. However, the present invention provides an engineered cell which is capable of producing a small molecule through the expression of one or more enzymes which are capable of synthesising the small molecule when expressed in combination in the cell.

The one or more enzymes may be referred to herein as a transgenic synthetic biology pathway. Suitably, the one or more enzymes comprise at least two, at least three, at least four or at least five enzymes. For example the transgenic synthetic biology pathway may comprise or consist of 2, 3, 4, 5 or more enzymes.

Accordingly, the cell of the present invention may encode a set of enzymes which when translated effect the stepwise conversion of a starting material in the cell to a therapeutic small molecule.

Suitably, the one or more enzymes are encoded one or more engineered polynucleotides. For example, the one or more enzymes may be encoded by one, two, three, four, five or more engineered polynucleotides.

In one embodiment, each enzyme of the transgenic synthetic biology pathway is encoded by a separate engineered polynucleotide.

The expression of each enzyme of the transgenic synthetic biology pathway may be controlled by a regulatory sequence such as a promoter. Suitably, the expression of each enzyme of the transgenic synthetic biology pathway may be controlled by related regulatory sequences so that each enzyme is expressed at the same time in the cell. Suitably, the expression of each enzyme of the transgenic synthetic biology pathway may be controlled by the same regulatory sequences so that each enzyme is expressed at the same time in the cell.

Suitably, the expression one or more enzymes of the transgenic synthetic biology pathway (for example a rate-limiting enzyme in the transgenic synthetic biology pathway) may be controlled by an inducible regulatory element so that production of the therapeutic small molecule can be induced in a controllable manner. Suitable embodiments for the inducible expression of one or more enzymes of the transgenic synthetic biology pathway are described herein.

Preferably, a plurality of enzymes of the transgenic synthetic biology pathway is encoded by an engineered polynucleotide. For example, two, three, four, five or more than five enzymes of a transgenic synthetic biology pathway may be encoded by the engineered polynucleotide.

An engineered polynucleotide encoding more than one enzyme (e.g. all required enzymes) of a transgenic synthetic biology pathway may be referred to as a transgenic synthetic biology pathway expression cassette.

Preferably, all of the enzymes required to form the transgenic synthetic biology pathway are encoded by a single engineered polynucleotide.

In embodiments where more than one enzyme is encoded by an engineered polynucleotide, the enzymes may be encoded as a single-reading frame under the control of the same regulatory elements (e.g. the same promoter).

Suitable, a co-expression site may be used to enable co-expression of the enzymes of the transgenic synthetic biology pathway as a single open-reading frame.

The co-expression site may be a sequence encoding a cleavage site, such that the engineered polynucleotide encodes the enzymes of the transgenic synthetic biology pathway joined by a cleavage site(s). Typically, a co-expression site is located between adjacent polynucleotide sequences which encode separate enzymes of the transgenic synthetic biology pathway.

Suitably, in embodiments where a plurality of co-expression sites are present in the engineered polynucleotide, the same co-expression site is used (i.e. the same co-expression site is present between each adjacent pair of nucleotide sequences encoding separate enzymes of the transgenic synthetic biology pathway.

Preferably, the co-expression site is a cleavage site. The cleavage site may be any sequence which enables the two polypeptides to become separated. The cleavage site may be self-cleaving, such that when the polypeptide is produced, it is immediately cleaved into individual peptides without the need for any external cleavage activity.

The term “cleavage” is used herein for convenience, but the cleavage site may cause the peptides to separate into individual entities by a mechanism other than classical cleavage. For example, for the Foot-and-Mouth disease virus (FM DV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a furin cleavage site.

Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al., 2001) as above.

The co-expression sequence may be an internal ribosome entry sequence (IRES). The co-expressing sequence may be an internal promoter.

Suitably, the engineered polynucleotide may be an operon. An operon is a functioning polynucleotide unit which comprises a plurality of genes under the control of a single promoter. The genes are transcribed together into an mRNA strand and either translated together in the cytoplasm, or undergo trans-splicing to create monocistronic mRNAs that are translated separately, i.e. several strands of mRNA that each encode a single gene product. The result of this is that the genes contained in the operon are either expressed together or not at all.

Therapeutic Small Molecule

The therapeutic small molecule may be any small molecule which is efficacious in the treatment of cancer.

“Therapeutic small molecule” is used herein according to its usual meaning to refer to a pharmaceutical molecule with a low molecular weight (e.g. less than 900 daltons).

Transgenic synthetic biology pathways which are suitable for producing a wide range of small molecules which may be used in the present invention are known in the art. By way of example the small molecule may be an alkaloid, terpenoid, flavonoid, polyketides or non-ribosomal peptides, sugar or sugar alcohol.

Alkaloids are nitrogen-containing compounds of low molecular weight produced by a large variety of organisms, including bacteria, fungi, plants, and animals. Most alkaloids are derived through decarboxylation of amino acids such as tryptophan, tyrosine, ornithine, histidine, and lysine, and possess important pharmacological activities. For example, sanguinarine has shown potential as an anticancer therapeutic, bisbenzyliso-quinoline alkaloid tetrandrine has immunomodulatory effects, and a number of indolocarbazole alkaloids have entered clinical trials for inhibiting neovascularization and as cancer treatments.

Alkaloids can be classified into a number of groups such as morphinane-, protoberberine-, ergot-, pyrrolizidine-, quinolizidine- and furanoquinoline-alkaloids according to the amino acids from which they originate.

Benzylisoquinoline alkaloids, such as sanguinarine, are synthesized from tyrosine via reticuline in Magnoliaceae, Ranunculaceae, Berberidaceae, Papaveraceae, and many other species. The early pathway from tyrosine to reticuline is common among many plant species, whereas there is more diversity in late pathways.

The therapeutic small molecule may be selected from a cytotoxic molecule; a cytostatic molecule; an agent which is capable of inducing differentiation of the tumour; and a proinflammatory molecule.

A cytotoxic molecule refers to a molecule which is directly toxic to a cell and is capable of inducing cell death. For example, a cytotoxic molecule may disrupt DNA synthesis, protein synthesis and/or metabolic processes within the cell.

Illustrative cytotoxic molecules include, but are not limited to, violacein, mycophenolic acid, terpenes/isoprenoids (e.g. geraniol, sesterterpenes such as ophiobolin derivatives; Taxol), triterpenoids (e.g. ginsenosides, oleanolic acid, ursolic acid, betulinic acid or protopanaxadiol), cyclosporin, Tacrolimus, Methotrexate, sanguinarine and fluorouracil.

The cytotoxic molecule may be selected from one of the following types: alkylators, such as cyclophosphamide; anthracyclines, such as daunorubicin; antimetabolites, such as cytarabine; vinca alkaloids, such as vincristine; and topoisomerase inhibitors, such as etoposide.

A cytostatic molecule refers to molecules which are capable of modulating cell cycle and cell growth, in particular molecules which are capable of inducing cell growth arrest. For example, all trans retinoic acid (ATRA) can induce differentiation of certain types of acute myeloid leukaemia.

Synthesis of Violacein

Suitably, the therapeutic small molecule may be violacein

Violacein is an indole derivative, isolated mainly from bacteria of the genus Chromobacterium. Violacein exhibits important anti-tumour properties—for example violacein has activity against MOLT-4 leukaemia, NCI-H460 non-small-cell lung cancer and KM12 colon-cancer cell lines.

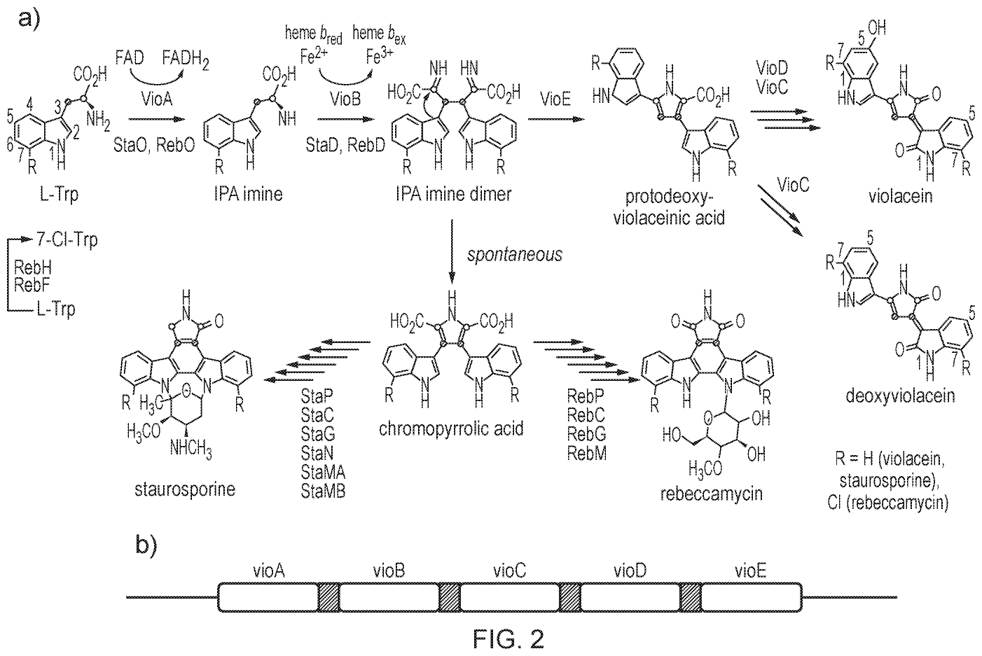

Violacein is formed by enzymatic condensation of two tryptophan molecules, requiring the action of five proteins (see FIG. 2). The genes required for its production may be referred to as vioABCDE (see August et al.; Journal of Molecular Microbiology and Biotechnology, vol. 2, no. 4, pp. 513-519, 2000—herein incorporated by reference) and have been cloned and expressed within other bacterial hosts, such as E. coli. The vioABCDE genes encode the enzymes VioA, VioB, VioC, VioD and VioE.

The one or more engineered polynucleotides may encode VioA, VioB, VioC, VioD and VioE such that the engineered cell of the present invention is capable of synthesising violacein from tryptophan.

The amino acid sequences for VioA, VioB, VioC, VioD and VioE are shown below as SEQ ID No. 1-5 respectively.

SEQ ID No. 1 - VioA
MKHSSDICIVGAGISGLTCASHLLDSPACRGLSLRIFDMQQEAGGRIRS
KMLDGKASIELGAGRYSPQLHPHFQSAMQHYSQKSEVYPFTQLKFKSHV
QQKLKRAMNELSPRLKEHGKESFLQFVSRYQGHDSAVGMIRSMGYDALF
LPDISAEMAYDIVGKHPEIQSVTDNDANQWFAAETGFAGLIQGIKAKVK
AAGARFSLGYRLLSVRTDGDGYLLQLAGDDGWKLEHRTRHLILAIPPSA
MAGLNVDFPEAWSGARYGSLPLFKGFLTYGEPWWLDYKLDDQVLIVDNP
LRKIYFKGDKYLFFYTDSEMANYWRGCVAEGEDGYLEQIRTHLASALGI
VRERIPQPLAHVHKYWAHGVEFCRDSDIDHPSALSHRDSGIIACSDAYT
EHCGWMEGGLLSAREASRLLLQRIAA
SEQ ID No. 2 - VioB
MSILDFPRIHFRGWARVNAPTANRDPHGHIDMASNTVAMAGEPFDLARH
PTEFHRHLRSLGPRFGLDGRADPEGPFSLAEGYNAAGNNHFSWESATVS
HVQWDGGEADRGDGLVGARLALWGHYNDYLRTTFNRARWVDSDPTRRDA
AQIYAGQFTISPAGAGPGTPWLFTADIDDSHGARWTRGGHIAERGGHFL
DEEFGLARLFQFSVPKDHPHFLFHPGPFDSEAWRRLQLALEDDDVLGLT
VQYALFNMSTPPQPNSPVFHDMVGVVGLWRRGELASYPAGRLLRPRQPG
LGDLTLRVNGGRVALNLACAIPFSTRAAQPSAPDRLTPDLGAKLPLGDL
LLRDEDGALLARVPQALYQDYWTNHGIVDLPLLREPRGSLTLSSELAEW
REQDWVTQSDASNLYLEAPDRRHGRFFPESIALRSYFRGEARARPDIPH
RIEGMGLVGVESRQDGDAAEWRLTGLRPGPARIVLDDGAEAIPLRVLPD
DWALDDATVEEVDYAFLYRHVMAYYELVYPFMSDKVFSLADRCKCETYA
RLMWQMCDPQNRNKSYYMPSTRELSAPKARLFLKYLAHVEGQARLQAPP
PAGPARIESKAQLAAELRKAVDLELSVMLQYLYAAYSIPNYAQGQQRVR
DGAWTAEQLQLACGSGDRRRDGGIRAALLEIAHEEMIHYLVVNNLLMAL
GEPFYAGVPLMGEAARQAFGLDTEFALEPFSESTLARFVRLEWPHFIPA
PGKSIADCYAAIRQAFLDLPDLFGGEAGKRGGEHHLFLNELTNRAHPGY
QLEVFDRDSALFGIAFVTDQGEGGALDSPHYEHSHFQRLREMSARIMAQ
SAPFEPALPALRNPVLDESPGCQRVADGRARALMALYQGVYELMFAMMA
QHFAVKPLGSLRRSRLMNAAIDLMTGLLRPLSCALMNLPSGIAGRTAGP
PLPGPVDTRSYDDYALGCRMLARRCERLLEQASMLEPGWLPDAQMELLD
FYRRQMLDLACGKLSREA
SEQ ID No. 3 - VioC
MKRAIIVGGGLAGGLTAIYLAKRGYEVHVVEKRGDPLRDLSSYVDVVSS
RAIGVSMTVRGIKSVLAAGIPRAELDACGEPIVAMAFSVGGQYRMRELK
PLEDFRPLSLNRAAFQKLLNKYANLAGVRYYFEHKCLDVDLDGKSVLIQ
GKDGQPQRLQGDMIIGADGAHSAVRQAMQSGLRRFEFQQTFFRHGYKTL
VLPDAQALGYRKDTLYFFGMDSGGLFAGRAATIPDGSVSIAVCLPYSGS
PSLTTTDEPTMRAFFDRYFGGLPRDARDEMLRQFLAKPSNDLINVRSST
FHYKGNVLLLGDAAHATAPFLGQGMNMALEDARTFVELLDRHQGDQDKA
FPEFTELRKVQADAMQDMARANYDVLSCSNPIFFMRARYTRYMHSKFPG
LYPPDMAEKLYFTSEPYDRLQQIQRKQNVWYKIGRVN
SEQ ID No. 4 - VioD
MKILVIGAGPAGLVFASQLKQARPLWAIDIVEKNDEQEVLGWGVVLPGR
PGQHPANPLSYLDAPERLNPQFLEDFKLVHHNEPSLMSTGVLLCGVERR
GLVHALRDKCRSQGIAIRFESPLLEHGELPLADYDLVVLANGVNHKTAH
FTEALVPQVDYGRNKYIWYGTSQLFDQMNLVFRTHGKDIFIAHAYKYSD
TMSTFIVECSEETYARARLGEMSEEASAEYVAKVFQAELGGHGLVSQPG
LGWRNFMTLSHDRCHDGKLVLLGDALQSGHFSIGHGTTMAVVVAQLLVK
ALCTEDGVPAALKRFEERALPLVQLFRGHADNSRVWFETVEERMHLSSA
EFVQSFDARRKSLPPMPEALAQNLRYALQR
SEQ ID No. 5 - VioE
MENREPPLLPARWSSAYVSYWSPMLPDDQLTSGYCWFDYERDICRIDGL
FNPWSERDTGYRLWMSEVGNAASGRTWKQKVAYGRERTALGEQLCERPL
DDETGPFAELFLPRDVLRRLGARHIGRRVVLGREADGWRYQRPGKGPST
LYLDAASGTPLRMVTGDEASRASLRDFPNVSEAEIPDAVFAAKR

An illustrative violacein single operon reading frame comprising the VioA, VioB, VioC, VioD and VioE polypeptides in frame with each other and separated by foot-and-mouth like 2A sequences is shown as SEQ ID NO: 6. In this sequence, the 2A peptide sequences are shown in bold and italic. A nucleic acid sequence which encodes the violacein ORF is shown as SEQ ID No. 7.

SEQ ID NO: 6 - Violacein ORF
MKHSSDICIVGAGISGLTCASHLLDSPACRGLSLRIFDMQQEAGGRIRSKMLDGKASIELGA
GRYSPQLHPHFQSAMQHYSQKSEVYPFTQLKFKSHVQQKLKRAMNELSPRLKEHGKESFL
QFVSRYQGHDSAVGMIRSMGYDALFLPDISAEMAYDIVGKHPEIQSVTDNDANQWFAAET
GFAGLIQGIKAKVKAAGARFSLGYRLLSVRTDGDGYLLQLAGDDGWKLEHRTRHLILAIPPS
AMAGLNVDFPEAWSGARYGSLPLFKGFLTYGEPWWLDYKLDDQVLIVDNPLRKIYFKGDK
YLFFYTDSEMANYWRGCVAEGEDGYLEQIRTHLASALGIVRERIPQPLAHVHKYWAHGVEF
CRDSDIDHPSALSHRDSGIIACSDAYTEHCGWMEGGLLSAREASRLLLQRIAA [symbol] [symbol]
MSILDFPRIHFRGWARVNAPTANRDPHGHIDMASNTVAMAGEPFDLAR
HPTEFHRHLRSLGPRFGLDGRADPEGPFSLAEGYNAAGNNHFSWESATVSHVQWDGGEA
DRGDGLVGARLALWGHYNDYLRTTFNRARWVDSDPTRRDAAQIYAGQFTISPAGAGPGTP
WLFTADIDDSHGARWTRGGHIAERGGHFLDEEFGLARLFQFSVPKDHPHFLFHPGPFDSE
AWRRLQLALEDDDVLGLTVQYALFNMSTPPQPNSPVFHDMVGVVGLWRRGELASYPAGR
LLRPRQPGLGDLTLRVNGGRVALNLACAIPFSTRAAQPSAPDRLTPDLGAKLPLGDLLLRDE
DGALLARVPQALYQDYWTNHGIVDLPLLREPRGSLTLSSELAEWREQDWVTQSDASNLYL
EAPDRRHGRFFPESIALRSYFRGEARARPDIPHRIEGMGLVGVESRQDGDAAEWRLTGLR
PGPARIVLDDGAEAIPLRVLPDDWALDDATVEEVDYAFLYRHVMAYYELVYPFMSDKVFSL
ADRCKCETYARLMWQMCDPQNRNKSYYMPSTRELSAPKARLFLKYLAHVEGQARLQAPP
PAGPARIESKAQLAAELRKAVDLELSVMLQYLYAAYSIPNYAQGQQRVRDGAWTAEQLQLA
CGSGDRRRDGGIRAALLEIAHEEMIHYLVVNNLLMALGEPFYAGVPLMGEAARQAFGLDTE
FALEPFSESTLARFVRLEWPHFIPAPGKSIADCYAAIRQAFLDLPDLFGGEAGKRGGEHHLF
LNELTNRAHPGYQLEVFDRDSALFGIAFVTDQGEGGALDSPHYEHSHFQRLREMSARIMA
QSAPFEPALPALRNPVLDESPGCQRVADGRARALMALYQGVYELMFAMMAQHFAVKPLG
SLRRSRLMNAAIDLMTGLLRPLSCALMNLPSGIAGRTAGPPLPGPVDTRSYDDYALGCRML
ARRCERLLEQASMLEPGWLPDAQMELLDFYRRQMLDLACGKLSREA [symbol] [symbol]
MKRAIIVGGGLAGGLTAIYLAKRGYEVHVVEKRGDPLRDLSSYVDVVSSRAIGVS
MTVRGIKSVLAAGIPRAELDACGEPIVAMAFSVGGQYRMRELKPLEDFRPLSLNRAAFQKLL
NKYANLAGVRYYFEHKCLDVDLDGKSVLIQGKDGQPQRLQGDMIIGADGAHSAVRQAMQS
GLRRFEFQQTFFRHGYKTLVLPDAQALGYRKDTLYFFGMDSGGLFAGRAATIPDGSVSIAV
CLPYSGSPSLTTTDEPTMRAFFDRYFGGLPRDARDEMLRQFLAKPSNDLINVRSSTFHYKG
NVLLLGDAAHATAPFLGQGMNMALEDARTFVELLDRHQGDQDKAFPEFTELRKVQADAMQ
DMARANYDVLSCSNPIFFMRARYTRYMHSKFPGLYPPDMAEKLYFTSEPYDRLQQIQRKQ
NVWYKIGRVN [symbol] [symbol] MKILVIGAGPAGLVFASQLKQARPLWAIDIV
EKNDEQEVLGWGVVLPGRPGQHPANPLSYLDAPERLNPQFLEDFKLVHHNEPSLMSTGVL
LCGVERRGLVHALRDKCRSQGIAIRFESPLLEHGELPLADYDLVVLANGVNHKTAHFTEALV
PQVDYGRNKYIVVYGTSQLFDQMNLVFRTHGKDIFIAHAYKYSDTMSTFIVECSEETYARARL
GEMSEEASAEYVAKVFQAELGGHGLVSQPGLGWRNFMTLSHDRCHDGKLVLLGDALQSG
HFSIGHGTTMAVVVAQLLVKALCTEDGVPAALKRFEERALPLVQLFRGHADNSRVWFETVE
ERMHLSSAEFVQSFDARRKSLPPMPEALAQNLRYALQR [symbol] [symbol] M
ENREPPLLPARWSSAYVSYWSPMLPDDQLTSGYCWFDYERDICRIDGLFNPWSERDTGY
RLWMSEVGNAASGRTWKQKVAYGRERTALGEQLCERPLDDETGPFAELFLPRDVLRRLG
ARHIGRRVVLGREADGWRYQRPGKGPSTLYLDAASGTPLRMVTGDEASRASLRDFPNVSE
AEIPDAVFAAKR
SEQ ID No. 7 - Violacein ORF DNA
ATGAAACACTCTTCTGATATTTGTATAGTTGGGGCAGGGATATCAGGCCTCACCTGTGC
TTCACACCTTCTTGATAGCCCAGCTTGCAGGGGCCTGTCACTTCGAATTTTTGACATGC
AACAGGAGGCCGGCGGACGGATCCGCTCTAAGATGCTTGATGGCAAGGCGTCTATCG
AACTCGGCGCCGGACGGTACTCTCCGCAACTTCACCCCCACTTCCAAAGTGCAATGCA
ACACTACAGTCAAAAATCCGAGGTCTACCCATTCACCCAATTGAAGTTCAAATCCCATGT
TCAACAGAAACTCAAACGGGCCATGAACGAACTGTCACCGCGCCTTAAGGAGCACGGA
AAGGAGAGCTTTCTCCAGTTTGTGTCTCGCTACCAGGGTCATGACTCCGCTGTAGGGA
TGATTAGGTCCATGGGGTATGATGCCCTCTTTCTCCCGGATATATCAGCTGAAATGGCT
TATGACATTGTTGGCAAGCATCCCGAAATTCAGTCTGTCACGGACAACGATGCCAACCA
GTGGTTTGCAGCAGAAACAGGCTTTGCGGGCCTTATACAGGGAATTAAAGCCAAAGTA
AAGGCCGCTGGTGCTCGATTCTCACTTGGCTATCGACTCCTCAGTGTTAGGACAGATG
GTGATGGCTATCTCTTGCAATTGGCCGGCGACGATGGTTGGAAGTTGGAGCACCGAAC
CCGCCACTTGATCCTCGCCATCCCACCTTCTGCAATGGCTGGACTTAACGTCGACTTCC
CTGAAGCTTGGTCAGGGGCACGATATGGCTCACTCCCTCTCTTCAAAGGGTTCCTTACT
TACGGAGAGCCTTGGTGGCTTGACTATAAGCTTGACGACCAGGTTCTCATTGTAGATAA
TCCGCTCAGGAAGATTTATTTCAAAGGCGACAAGTACCTCTTCTTCTATACTGATTCTGA
GATGGCTAACTATTGGAGGGGCTGCGTAGCGGAAGGGGAGGACGGGTATCTGGAACA
AATACGAACCCACCTGGCCAGTGCCCTTGGCATAGTACGGGAGCGGATACCACAGCCT
CTCGCTCATGTGCACAAGTATTGGGCGCATGGTGTCGAATTCTGCCGCGACTCTGACA
TCGATCACCCCTCCGCCCTGAGTCACAGGGATTCAGGTATTATTGCTTGCAGCGATGC
GTATACCGAACATTGCGGTTGGATGGAAGGAGGTCTGCTGTCTGCCCGAGAAGCCTCC
CGACTGCTCCTTCAGAGAATCGCGGCAAGAGCAGAAGGGCGGGGGAGCCTTCTTACA
TGTGGAGACGTGGAGGAAAATCCAGGACCTATGTCAATTCTGGATTTTCCGCGCATCCA
TTTTAGAGGCTGGGCGAGAGTCAACGCTCCAACAGCCAACCGGGACCCGCATGGCCA
CATCGATATGGCGTCTAACACAGTGGCAATGGCAGGGGAGCCATTCGATCTTGCTAGA
CACCCGACAGAGTTCCATCGACATTTGCGAAGTTTGGGACCGCGGTTCGGCCTCGACG
GGAGAGCAGACCCGGAAGGTCCGTTCTCTCTTGCGGAGGGGTATAATGCCGCAGGCA
ACAATCACTTTTCTTGGGAATCTGCTACGGTATCCCATGTGCAATGGGATGGGGGTGAA
GCAGACCGAGGTGATGGGCTTGTCGGCGCAAGACTCGCACTGTGGGGACACTATAAC
GATTACTTGCGCACCACCTTCAACCGAGCGCGATGGGTCGACAGCGATCCGACCCGG
CGGGATGCCGCTCAGATATATGCTGGGCAATTTACCATTTCCCCAGCCGGGGCCGGGC
CAGGGACGCCATGGTTGTTCACGGCAGACATTGATGACTCCCATGGCGCCCGGTGGA
CCCGAGGAGGTCACATCGCGGAAAGGGGGGGTCATTTTTTGGACGAGGAATTTGGCCT
GGCAAGACTTTTTCAATTCTCCGTTCCGAAAGACCACCCACATTTTCTTTTCCATCCTGG
ACCTTTCGATTCCGAAGCTTGGAGAAGGCTGCAACTGGCGTTGGAGGACGACGATGTA
CTGGGCCTGACTGTCCAGTACGCTCTTTTTAACATGAGTACTCCACCACAACCCAACAG
CCCAGTCTTCCACGATATGGTAGGAGTGGTTGGGTTGTGGAGAAGAGGAGAGCTCGCA
AGCTATCCCGCGGGACGACTGCTTCGCCCCCGACAGCCGGGGCTCGGAGATCTTACG
CTTAGAGTCAACGGCGGCAGAGTTGCTCTTAACCTCGCATGCGCAATTCCATTCTCTAC
TCGGGCAGCTCAGCCCTCCGCTCCGGATAGGTTGACACCTGACCTCGGAGCAAAACTG
CCGCTCGGCGATCTTCTCCTTAGGGACGAGGACGGTGCGCTGCTGGCCAGGGTACCC
CAAGCGCTTTACCAAGATTACTGGACGAACCATGGAATAGTGGACTTGCCTCTCCTTCG
GGAACCTAGAGGCTCACTTACATTGTCCTCCGAGCTGGCAGAGTGGAGGGAACAGGAC
TGGGTTACACAAAGCGACGCGTCCAATTTGTATCTTGAAGCTCCTGACCGGCGCCATG
GGCGATTTTTTCCGGAAAGTATAGCGCTCAGGAGCTATTTCAGAGGTGAAGCAAGGGC
GCGACCGGACATTCCCCATCGGATTGAAGGCATGGGCCTCGTGGGGGTCGAGAGCCG
GCAGGACGGGGATGCCGCAGAATGGCGCTTGACAGGATTGAGGCCGGGTCCGGCAA
GGATTGTGCTGGATGATGGGGCCGAGGCAATTCCATTGCGAGTACTGCCCGATGACTG
GGCTTTGGACGATGCGACTGTCGAAGAAGTAGATTACGCGTTTCTTTACAGGCACGTTA
TGGCTTACTACGAACTGGTATACCCATTTATGAGCGATAAGGTATTCTCACTGGCCGAC
CGATGCAAATGCGAGACGTACGCGCGCCTGATGTGGCAAATGTGTGATCCTCAGAATC
GCAATAAAAGTTACTACATGCCGAGTACGCGCGAGCTCAGCGCACCAAAGGCTCGCCT
GTTTCTGAAGTACTTGGCCCATGTGGAAGGGCAGGCGAGGTTGCAAGCTCCCCCACCA
GCCGGGCCCGCCAGAATAGAAAGTAAAGCCCAATTGGCCGCAGAGTTGCGCAAAGCC
GTCGATTTGGAACTCTCCGTCATGCTTCAATATCTCTACGCAGCGTATTCTATACCGAAC
TACGCACAGGGTCAACAAAGAGTCAGAGACGGTGCGTGGACCGCCGAACAGCTTCAA
CTTGCATGCGGTAGCGGTGATAGGCGAAGGGACGGTGGTATACGCGCGGCATTGTTG
GAAATTGCCCACGAAGAAATGATACATTACCTCGTGGTCAACAATCTTCTCATGGCGCT
GGGCGAACCATTCTATGCCGGCGTGCCCCTTATGGGGGAAGCAGCTAGGCAAGCTTTC
GGCCTGGACACAGAATTTGCTCTTGAGCCGTTTTCCGAGTCAACTTTGGCACGATTCGT
CCGGTTGGAATGGCCACACTTTATCCCAGCCCCAGGAAAGAGTATAGCGGATTGTTAT
GCTGCAATCCGACAGGCTTTTCTTGATCTCCCCGATCTCTTTGGCGGTGAGGCCGGGA
AACGAGGTGGCGAGCACCACCTCTTCTTGAATGAATTGACCAACCGCGCACACCCGGG
TTACCAACTGGAAGTATTTGATAGGGATAGCGCGTTGTTTGGAATAGCGTTTGTCACCG
ATCAAGGTGAAGGCGGTGCACTCGACAGTCCGCACTATGAACACTCCCACTTTCAGCG
GTTGCGGGAAATGAGCGCACGGATAATGGCTCAATCCGCTCCCTTCGAACCTGCCCTT
CCGGCCCTCAGAAACCCCGTTCTCGATGAGAGCCCAGGCTGCCAACGGGTGGCCGAC
GGGCGCGCACGCGCGCTGATGGCACTGTACCAGGGGGTGTACGAACTGATGTTCGCA
ATGATGGCTCAGCACTTTGCTGTAAAACCGCTCGGGAGTCTTCGAAGGTCCAGGTTGA
TGAATGCCGCAATTGATTTGATGACCGGGCTCCTCCGCCCTTTGTCATGTGCTCTCATG
AATTTGCCTTCAGGTATAGCGGGGCGCACCGCAGGACCGCCACTTCCAGGACCCGTTG
ACACGCGAAGCTACGACGATTATGCCCTGGGCTGCCGAATGCTGGCACGACGCTGCG
AACGACTGCTTGAGCAAGCGTCCATGCTGGAACCCGGATGGCTTCCCGACGCCCAGAT
GGAACTCCTGGATTTCTATCGACGCCAGATGCTGGATCTTGCGTGCGGGAAGCTGAGT
AGGGAGGCGCAGTGTACTAACTATGCTCTGTTGAAATTGGCTGGGGATGTCGAATCCA
ATCCAGGCCCTATGAAACGAGCAATCATTGTCGGCGGCGGCCTCGCCGGTGGCCTGA
CAGCCATCTATTTGGCTAAACGCGGGTATGAGGTCCATGTAGTAGAGAAGAGAGGTGA
TCCTTTGCGAGATTTGAGCAGCTATGTTGACGTGGTATCTTCCCGGGCCATCGGTGTCA
GTATGACGGTCAGAGGCATAAAATCCGTGTTGGCGGCCGGTATCCCACGCGCCGAACT
GGATGCTTGTGGCGAGCCAATTGTAGCAATGGCATTCTCCGTAGGCGGGCAATACCGA
ATGCGGGAACTTAAACCGCTCGAGGATTTCCGGCCACTGTCATTGAATCGGGCTGCGT
TCCAAAAACTGCTTAATAAATACGCAAACCTTGCAGGCGTTAGGTATTATTTCGAGCACA
AGTGTCTCGATGTCGATTTGGACGGGAAAAGTGTTCTGATTCAAGGAAAAGACGGGCA
ACCGCAGCGCCTTCAGGGTGACATGATAATAGGCGCGGACGGCGCGCACAGCGCCGT
ACGACAGGCCATGCAATCTGGACTCCGGCGGTTTGAATTCCAGCAAACATTTTTCCGCC
ATGGGTATAAGACTTTGGTTCTGCCTGATGCGCAAGCTTTGGGGTATCGGAAAGATACG
CTCTATTTCTTTGGGATGGATAGTGGAGGGCTTTTCGCCGGACGCGCTGCTACGATTC
CCGACGGAAGTGTCTCAATAGCAGTCTGTCTTCCGTACAGTGGATCCCCGAGCCTTAC
GACTACGGATGAACCGACCATGCGGGCGTTTTTCGACCGCTACTTCGGAGGTTTGCCG
AGAGATGCTCGGGACGAAATGCTCAGGCAATTCCTTGCCAAACCGAGTAACGATTTGAT
CAACGTGCGGTCTTCCACATTTCACTATAAAGGTAACGTGCTGTTGCTGGGCGACGCA
GCCCACGCAACAGCACCGTTCCTGGGGCAAGGGATGAATATGGCATTGGAAGACGCG
AGAACGTTCGTCGAGTTGCTTGATCGCCACCAAGGTGATCAGGATAAAGCGTTTCCGG
AATTTACAGAGCTTAGGAAGGTTCAAGCCGATGCTATGCAAGACATGGCACGAGCGAA
CTATGATGTGCTCAGCTGTAGTAACCCGATCTTTTTTATGAGAGCAAGATATACGAGGT
ACATGCATAGTAAATTCCCAGGTCTGTACCCCCCCGATATGGCTGAGAAACTCTATTTC
ACGTCTGAGCCGTATGATCGATTGCAACAGATCCAGCGAAAACAAAATGTATGGTATAA
GATTGGTCGCGTTAATCGAGCAGAAGGGCGAGGGTCACTGTTGACATGTGGTGACGTG
GAAGAGAACCCCGGCCCTATGAAGATCCTCGTCATCGGCGCGGGACCAGCCGGTTTG
GTGTTTGCGTCCCAACTTAAACAGGCGAGGCCCCTGTGGGCGATAGATATCGTCGAAA
AAAACGATGAACAAGAGGTGCTTGGATGGGGGGTGGTCTTGCCTGGTAGACCGGGTC
AGCACCCTGCGAATCCGCTTAGCTACCTCGACGCGCCCGAGAGGCTGAACCCTCAGTT
CCTTGAAGACTTCAAACTGGTGCATCATAATGAACCAAGTCTCATGTCTACCGGAGTAC
TTTTGTGCGGGGTCGAGAGACGGGGCCTGGTCCATGCTCTGCGGGATAAGTGCAGGT
CCCAAGGTATAGCTATTAGGTTTGAAAGTCCATTGCTTGAACATGGCGAACTTCCCTTG
GCGGATTATGATCTTGTGGTACTCGCAAACGGAGTGAACCATAAGACCGCGCATTTTAC
CGAGGCTCTGGTTCCTCAGGTCGACTATGGTCGAAACAAGTACATTTGGTACGGCACC
TCCCAACTTTTCGATCAAATGAACCTGGTATTTAGGACGCACGGCAAAGACATTTTCATT
GCTCATGCGTATAAATACTCCGACACCATGTCCACGTTTATTGTCGAGTGCTCTGAGGA
GACGTACGCTAGGGCCCGGCTGGGCGAAATGAGTGAGGAAGCATCAGCAGAATACGT
CGCCAAGGTTTTCCAAGCAGAACTCGGAGGGCATGGGCTGGTAAGCCAACCCGGATT
GGGATGGAGGAACTTCATGACTCTTAGCCACGATCGCTGCCATGACGGAAAACTCGTG
TTGTTGGGGGACGCACTCCAGAGCGGTCACTTTAGTATTGGACACGGTACCACGATGG
CTGTTGTGGTAGCACAGTTGCTTGTCAAAGCGTTGTGCACAGAGGATGGTGTACCCGC
AGCGCTTAAGCGCTTCGAGGAGAGGGCTCTGCCCCTGGTTCAACTTTTCCGCGGTCAT
GCGGACAACAGCCGGGTATGGTTTGAAACAGTTGAGGAGCGAATGCACTTGTCCTCCG
CTGAATTTGTCCAAAGCTTTGATGCCCGCCGGAAAAGTCTTCCGCCTATGCCTGAAGCG
CTTGCTCAGAATCTTCGATATGCCCTCCAGAGGAGGGCCGAGGGGCGGGGCTCACTT
CTTACGTGCGGTGACGTAGAAGAAAATCCCGGGCCTATGGAAAACCGGGAACCTCCCT
TGTTGCCAGCACGGTGGTCCTCCGCATATGTCTCCTACTGGTCACCGATGTTGCCAGA
CGATCAGCTGACCTCAGGGTACTGTTGGTTTGATTATGAGAGAGACATCTGCAGAATTG
ACGGTCTTTTTAACCCCTGGTCTGAGAGAGATACCGGTTACAGACTGTGGATGTCTGAA
GTAGGGAATGCAGCGAGTGGTAGGACCTGGAAGCAAAAAGTGGCATACGGCAGGGAG
CGAACGGCTTTGGGAGAACAGCTTTGCGAGCGACCATTGGATGACGAAACAGGCCCCT
TTGCCGAGTTGTTCCTGCCACGAGACGTATTGCGCAGACTTGGAGCACGACATATAGG
ACGCCGGGTAGTTCTGGGCAGGGAAGCCGATGGATGGAGATATCAGCGACCAGGAAA
AGGGCCAAGTACCCTGTATCTGGATGCAGCCAGCGGGACCCCACTTCGGATGGTCACT
GGAGACGAAGCGAGTCGCGCTTCCTTGAGGGATTTTCCCAACGTTTCCGAAGCGGAGA
TACCGGATGCTGTTTTTGCCGCCAAGCGC

The one or more enzymes which are capable of synthesising a therapeutic small molecule when expressed in combination in the cell may comprise one or more of the sequences shown as SEQ ID NO: 1 to 6, or a variant thereof having at least 80, 85, 90, 95, 98 or 99% sequence identity, provided that the variant VioA, VioB, VioC, VioD and/or VioE polypeptides retain the capacity to provide the required to form violacein from tryptophan in a cell.

The percentage identity between two polypeptide sequences may be readily determined by programs such as BLAST, which is freely available at http://blast.ncbi.nlm.nih.gov. Suitably, the percentage identity is determined across the entirety of the reference and/or the query sequence.

Synthesis of Geranyl Diphosphate Derived Terpenoids

The therapeutic small molecule may be a terpenoid.

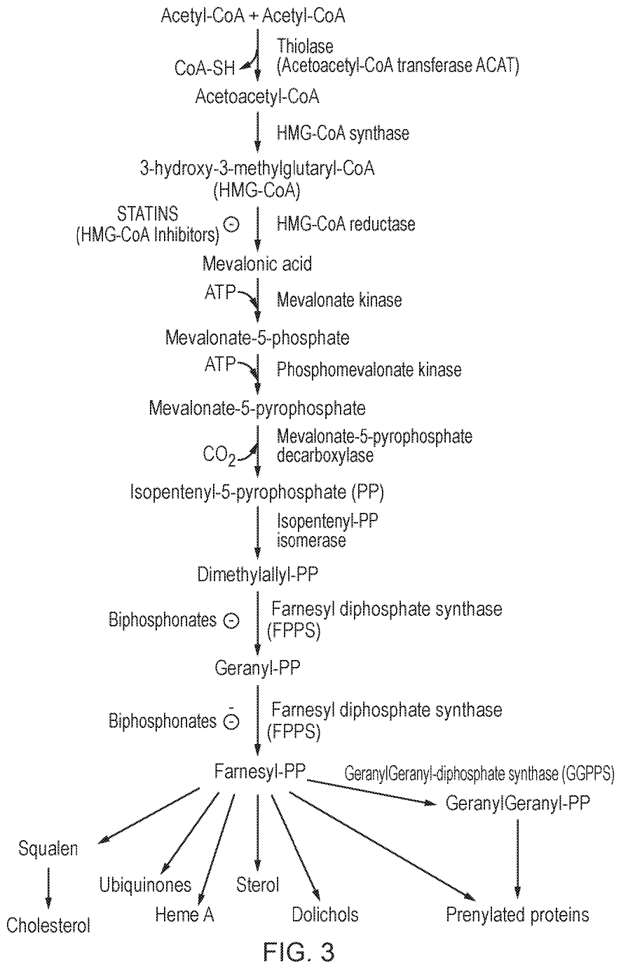

Terpenes constitute the largest group of secondary metabolites and are synthesized by all known organismal groups. Terpenes (or isoprenoids) have a wide range of applications but many possess anti-cancer properties. All terpenes are synthesized from two 5-carbon building blocks, isopentenyl phosphate (IDP) and demethylallyl diphosphate (DMADP). These building blocks are synthesized by two pathways. In humans, the mevalonate pathway is used and the final products are utilised for a variety of functions including cholesterol synthesis and precursors of protein prenylation (see FIG. 3).

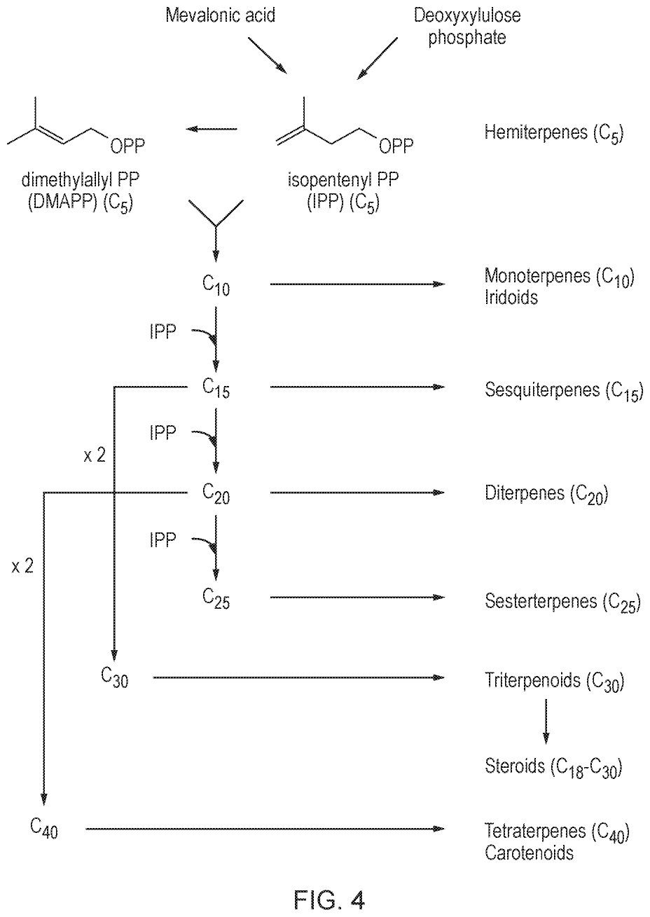

IDP and DMADP are combined by a variety of enzymes to produce a number of intermediates of differing five carbon combinations such as geranyl diphosphate (010), geranygeranyl diphosphate (C20) and squalene (C30) (see FIG. 4).

These combinations are the substrates for a wide range of terpene synthases which result in production of a huge variety of terpenoid products.

Further synthesis of more complex isoprenoids can also be achieved by expression of multiple enzymes in the engineered cell. Simple isoprenoids may be synthesized from mevalonate pathway precursors using a single enzymatic step.

For example, geraniol, a monoterpenoid synthesized by many plant species, is a major component of rose oil and has been shown to possess anti-cancer functions. Geraniol can be synthesized in yeast cells from geranyl diphosphate by expression of a single geraniol synthase gene from Valeriana officinalis (Zhao, J. et al.; (2016); App. Microbiol. and Biotech. 100, 4561-4571—incorporated herein by reference).

Accordingly, the one or more enzymes for use in the present invention may comprise a geraniol synthase enzyme. An illustrative geraniol synthase from Valeriana officinalis is shown as SEQ ID NO: 8 (corresponding to UniProt Accession Number—KF951406).

SEQ ID NO: 8
MITSSSSVRSLCCPKTSIISGKLLPSLLLTNVINVSNGTSSRACVSMSS
LPVSKSTASSIAAPLVRDNGSALNFFPQAPQVEIDESSRIMELVEATRR
TLRNESSDSTEKMRLIDSLQRLGLNHHFEQDIKEMLQDFANEHKNTNQD
LFTTSLRFRLLRHNGFNVTPDVFNKFTEENGKFKESLGEDTIGILSLYE
ASYLGGKGEEILSEAMKFSESKLRESSGHVAXHIRRQIFQSLELPRHLR
MARLESRRYIEEDYSNEIGADSSLLELAKLDFNSVQALHQMELTEISRW
WKQLGLSDKLPFARDRPLECFLWTVGLLPEPKYSGCRIELAKTIAVLLV
IDDIFDTYGSYDQLILFTNAIRRWDLDAMDELPEYMKICYMALYNTTNE
ICYKVLKENGWSVLPYLERTWIDMVEGFMLEAKWLNSGEQPNLEAYIEN
GVTTAGSYMALVHLFFLIGDGVNDENVKLLLDPYPKLFSSAGRILRLWD
DLGTAKEEQERGDVSSSIQLYMKEKNVRSESEGREGIVEIIYNLWKDMN
GELIGSNALPQAIIETSFNMARTSQVVYQHEDDTYFSSVDNYVQSLFFT
PVSVSV

The geraniol synthase may comprise the sequence shown as SEQ ID NO: 8 or a variant thereof having at least 80, 85, 90, 95, 98 or 99% sequence identity, provided that the variant sequence retains the capacity to produce geraniol from geranyl diphospate. The capacity of a variant enzyme to synthesise geranoil may be analysed using, for example, high performance liquid chromatography (HPLC) or mass spectroscopy.

More complex sesterterpenes such as ophiobolin derivatives, many of which have potent cytotoxic activities, can be synthesized using a single gene in Aspergillus sp. (Chai et al; (2016); Sci. Reports; 6, 1-11—incorporated herein by reference).

Accordingly, the one or more enzymes for use in the present invention may comprise a ophiobolin F synthase enzyme. An illustrative ophiobolin F synthase from Aspergillus clavatus is shown as SEQ ID NO: 9 (corresponding to UniProt Accession Number—A18C3).

SEQ ID NO: 9
MACKYSTLIDSSLYDREGLCPGIDLRRHVAGELEEVGAFRAQEDWRRLV
GPLPKPYAGLLGPDFSFITGAVPECHPDRMEIVAYALEFGFMHDDVIDT
DVNHASLDEVGHTLDQSRTGKIEDKGSDGKRQMVTQIIREMMAIDPERA
MTVAKSWASGVRHSSRRKEDTNFKALEQYIPYRALDVGYMLWHGLVTFG
CAITIPNEEEEEAKRLIIPALVQASLLNDLFSFEKEKNDANVQNAVLIV
MNEHGCSEEEARDILKKRIRLECANYLRNVKETNARADVSDELKRYINV
MQYTLSGNAAWSTNCPRYNGPTKFNELQLLRSEHGLAKYPSRWSQENRT
SGLVEGDCHESKPNELKRKRNGVSVDDEMRTNGTNGAKKPAHVSQPSTD
SIVLEDMVQLARTCDLPDLSDTVILQPYRYLTSLPSKGFRDQAIDSINK
WLKVPPKSVKMIKDVVKMLHSASLMLDDLEDNSPLRRGKPSTHSIYGMA
QTVNSATYQYITATDITAQLQNSETFHIFVEELQQLHVGQSYDLYWTHN
TLCPTIAEYLKMVDMKTGGLFRMLTRMMIAESPVVDKVPNSDMNLFSCL
IGRFFQIRDDYQNLASADYAKAKGFAEDLDEGKYSFTLIHCIQTLESKP
ELAGEMMQLRAFLMKRRHEGKLSQEAKQEVLVTMKKTESLQYTLSVLRE
LHSELEKEVENLEAKFGEENFTLRVMLELLKV

The ophiobolin F synthase may comprise the sequence shown as SEQ ID NO: 9 or a variant thereof having at least 80, 85, 90, 95, 98 or 99% sequence identity, provided that the variant sequence retains the capacity to produce an ophiobolin from dimethylallyl diphosphate (DMAPP), Geranyl diphosphate, farnesyl diphosphate or geranylgeranyl diphosphate.

Geraniol and ophiobolins are a relatively simple isoprenoid, but their synthesis demonstrates the feasibility of synthesizing more complex isoprenoids using multiple enzymes. A further example of a terpene derivative is Taxol, a complex tricyclic diterpene, requiring up to 19 enzymes to synthesize from IDP and DMADP precursors required for geraniol synthesis. This synthetic pathway and the enzymes involved are reviewed in Croteau et al (2006) Taxol biosynthesis and molecular genetics Phytochem Rev. 5:75-97.

Synthesis of Triterpenoids from Squalene

The therapeutic small molecule may be a triterpenoid.

Cholesterol is a cellular product derived from the mevalonate pathway requiring similar precursors to prenylation precursors, but enzymes directing the synthesis of squalene divert from the pathway to produce cholesterol (FIG. 3). Squalene is a triterpene and is a precursor for the synthesis of a wide variety of triterpene derived compounds (FIG. 5) many of which have anticancer activity.

By expression of four plant derived enzymes it has been possible to produce complex ginsenosides in yeast (Wang, P. et al.; (2015); Metabolic Engineering. 29, 97-105—incorporated herein by reference). In addition to ginsenosides having anti-cancer activity, precursor compounds such as oleanolic acid or protopanaxadiol have anticancer properties.

Accordingly, the one or more enzymes for use in the present invention may comprise a group of enzymes capable of producing ginsenosides. An illustrative group of four enzymes capable of producing ginsenosides are shown as SEQ ID NO: 10-13.

SEQ ID NO: 10 - Protein sequence of Dammarenediol 12-hydroxylase from Panax
ginseng (Uniprot H2DH16)
MAAAMVLFFSLSLLLLPLLLLFAYFSYTKRIPQKENDSKAPLPPGQTGWPLIGETLNYLSCVKSGVSENFVKYRK
EKYSPKVFRTSLLGEPMAILCGPEGNKFLYSTEKKLVQVWFPSSVEKMFPRSHGESNADNFSKVRGKMMFLLKVD
GMKKYVGLMDRVMKQFLETDWNRQQQINVHNTVKKYTVTMSCRVFMSIDDEEQVTRLGSSIQNIEAGLLAVPINI
PGTAMNRAIKTVKLLTREVEAVIKQRKVDLLENKQASQPQDLLSHLLLTANQDGQFLSESDIASHLIGLMQGGYT
TLNGTITFVLNYLAEFPDVYNQVLKEQVEIANSKHPKELLNWEDLRKMKYSWNVAQEVLRIIPPGVGTFREAITD
FTYAGYLIPKGWKMHLIPHDTHKNPTYFPSPEKFDPTRFEGNGPAPYTFTPFGGGPRMCPGIEYARLVILIFMHN
VVTNFRWEKLIPNEKILTDPIPRFAHGLPIHLHPHN
SEQ ID NO: 11 - Protein sequence of UGTPg45 from Panax ginseng (Uniprot
A0A0D5ZDC8)
MEREMLSKTHIMFIPFPAQGHMSPMMQFAKRLAWKGLRITIVLPAQIRDFMQITNPLINTECISFDFDKDDGMPY
SMQAYMGVVKLKVTNKLSDLLEKQRTNGYPVNLLVVDSLYPSRVEMCHQLGVKGAPFFTHSCAVGAIYYNARLGK
LKIPPEEGLTSVSLPSIPLLGRDDLPIIRTGTFPDLFEHLGNQFSDLDKADWIFFNTFDKLENEEAKWLSSQWPI
TSIGPLIPSMYLDKQLPNDKDNGINFYKADVGSCIKWLDAKDPGSVVYASFGSVKHNLGDDYMDEVAWGLLHSKY
HFIWVVIESERTKLSSDFLAEAEAEEKGLIVSWCPQLQVLSHKSIGSFMTHCGWNSTVEALSLGVPMVALPQQFD
QPANAKYIVDVWQIGVRVPIGEEGVVLRGEVANCIKDVMEGEIGDELRGNALKWKGLAVEAMEKGGSSDKNIDEF
ISKLVSS
SEQ ID NO: 12 - Protein sequence of NADPH-Cytochrome P450 reductase2 from
Arabidopsis thaliana (Uniprot Q9SUM3)
MSSSSSSSTSMIDLMAAIIKGEPVIVSDPANASAYESVAAELSSMLIENRQFAMIVTTSIAVLIGCIVMLVWRRS
GSGNSKRVEPLKPLVIKPREEEIDDGRKKVTIFFGTQTGTAEGFAKALGEEAKARYEKTRFKIVDLDDYAADDDE
YEEKLKKEDVAFFFLATYGDGEPTDNAARFYKWFTEGNDRGEWLKNLKYGVFGLGNRQYEHFNKVAKVVDDILVE
QGAQRLVQVGLGDDDQCIEDDFTAWREALWPELDTILREEGDTAVATPYTAAVLEYRVSIHDSEDAKFNDINMAN
GNGYTVFDAQHPYKANVAVKRELHTPESDRSCIHLEFDIAGSGLTYETGDHVGVLCDNLSETVDEALRLLDMSPD
TYFSLHAEKEDGTPISSSLPPPFPPCNLRTALTRYACLLSSPKKSALVALAAHASDPTEAERLKHLASPAGKDEY
SKWVVESQRSLLEVMAEFPSAKPPLGVFFAGVAPRLQPRFYSISSSPKIAETRIHVTCALVYEKMPTGRIHKGVC
STWMKNAVPYEKSENCSSAPIFVRQSNFKLPSDSKVPIIMIGPGTGLAPFRGFLQERLALVESGVELGPSVLFFG
CRNRRMDFIYEEELQRFVESGALAELSVAFSREGPTKEYVQHKMMDKASDIWNMISQGAYLYVCGDAKGMARDVH
RSLHTIAQEQGSMDSTKAEGFVKNLQTSGRYLRDVW
SEQ ID NO: 13 Protein sequence of Dammarenediol II Synthase from Panax
ginseng (Uniprot Q08IT1)
MWKQKGAQGNDPYLYSTNNFVGRQYWEFQPDAGTPEEREEVEKARKDYVNNKKLHGIHPCSDMLMRRQLIKESGI
DLLSIPPLRLDENEQVNYDAVTTAVKKALRLNRAIQAHDGHWPAENAGSLLYTPPLIIALYISGTIDTILTKQHK
KELIRFVYNHQNEDGGWGSYIEGHSTMIGSVLSYVMLRLLGEGLAESDDGNGAVERGRKWILDHGGAAGIPSWGK
TYLAVLGVYEWEGCNPLPPEFWLFPSSFPFHPAKMWIYCRCTYMPMSYLYGKRYHGPITDLVLSLRQEIYNIPYE
QIKWNQQRHNCCKEDLYYPHTLVQDLVWDGLHYFSEPFLKRWPFNKLRKRGLKRVVELMRYGATETRFITTGNGE
KALQIMSWWAEDPNGDEFKHHLARIPDFLWIAEDGMTVQSFGSQLWDCILATQAIIATNMVEEYGDSLKKAHFFI
KESQIKENPRGDFLKMCRQFTKGAWTFSDQDHGCVVSDCTAEALKCLLLLSQMPQDIVGEKPEVERLYEAVNVLL
YLQSRVSGGFAVWEPPVPKPYLEMLNPSEIFADIVVEREHIECTASVIKGLMAFKCLHPGHRQKEIEDSVAKAIR
YLERNQMPDGSWYGFWGICFLYGTFFTLSGFASAGRTYDNSEAVRKGVKFFLSTQNEEGGWGESLESCPSEKFTP
LKGNRTNLVQTSWAMLGLMFGGQAERDPTPLHRAAKLLINAQMDNGDFPQQEITGVYCKNSMLHYAEYRNIFPLW
ALGEYRKRVWLPKHQQLKI

The transgenic synthetic biology pathway capable of producing ginsenosides may comprise one or more of the amino acids sequence shown as SEQ ID NO: 10 to 13 or a variant thereof having at least 80% sequence identity. For example, the transgenic synthetic biology pathway capable of producing ginsenosides may comprise at least two, at least three or all four of the amino acids sequence shown as SEQ ID NO: 10 to 13 or a variant thereof having at least 80% sequence identity.

The variant of one of the sequences shown as SEQ ID NO: 10 to 13 may have at least 80, 85, 90, 95, 98 or 99% sequence identity, provided that the variant sequence retains the functional activity of the corresponding enzyme having the reference sequence shown as one of SEQ ID NO: 10 to 13.

Expression of a limited number of plant genes thus enables production of a large number of anticancer compounds. Engineering of further triterpene modifying enzymes will enable production of a huge variety of more complex isoprenoids.

Sensitivity to the Therapeutic Small Molecule

In some embodiments the engineered cell of the present invention is further engineered to have reduced sensitivity to the therapeutic small molecule produced by the transgenic synthetic biology pathway.

As used herein, “reduced sensitivity” means that the engineered cell of the present invention is less susceptible to, for example, a cytotoxic effect of the therapeutic small molecule compared to an equivalent control cell which expresses (i) a chimeric antigen receptor (CAR) or a transgenic T-cell receptor (TCR); and (ii) one or more engineered polynucleotides which encode one or more enzymes which are capable of synthesising a therapeutic small molecule when expressed in combination in the cell but which control cell has not been engineered to have reduced sensitivity to the therapeutic small molecule.

Suitably, the cell of the present invention may be at least 5%, at least 10%, at least 15%, at least 20%, at least 30%, at least 40% or at least 50% less susceptible to the effects of the small molecule compared to an equivalent control cell which has not been engineered to have reduced sensitivity to the therapeutic small molecule.

The effects of the small molecule may be determined using methods and assays which are known in the art. By way of example, the effect of the small molecule may be determined using cell death assays such as flow cytometric detection of Annexin V upregulation or 7AAD staining. Differentiation can also be assessed by flow-cytometry by using appropriate lineage markers for the tumour in question. Quiescence of the tumour can be determined by measuring cell growth by simple counting or tritiated thymidine incorporation. More detailed effects of the small molecule on the tumour can be determined by RNAseq analysis.

The cell of the present invention may be engineered to have reduced sensitivity to the therapeutic small molecule by introducing a mutation which provides resistance to the relevant therapeutic small molecule.

Suitable drug resistance mechanisms and mutations are known in the art and are summarised by Zahreddine et al., for example (Frontiers in Pharmacology; 2013; 4(28); 1-8; herein incorporated by reference).

Methods for introducing a polynucleotide encoding a protein comprising a resistance mutation are known in the art and include, for example, transfer to a cell using retroviral vectors. Methods for introducing a relevant mutation into a wild-type polypeptide sequence are also known in the art and include, but are not limited to, site directed mutagenesis.

Suitable combinations of therapeutic small molecules and resistance mutations include, but are not limited to, those listed Table 2 below:

TABLE 2

| | | Illustrative Resistance | |
|---|---|---|---|
| Small Molecule | Target Protein | Mutation | Reference |
| Mycophenolic Acid | Ionsine monophosphate | IMPDH2^{IY} | Jonnalagadda et al. |
| | dehydrogenase 2 | T333I | (PLoS ONE8(6); |
| | | S351Y | (2013); e65519. |
| Antithymidylates | Dihydrofolate reductase | L22F | Rushworth et al. |
| | | F31S | (Gene Therapy (2016); |
| | Thymidylate synthase | T51S | 23; 119-128) |
| | | G52S | |
| Tacrolimus | Calcineurin A/B | CnAL T351E; L354A | Brewin et al. Blood |
| | | CnB L124T; K125-LA-Ins | 114, 4792-4803 |
| Cyclosporin | Calcineurin A/B | CnA: V314R; Y341F | (2009). |
| | | CnB L124T; K125-LA-Ins | |

Inducing Expression of the Therapeutic Small Molecule

In some embodiments expression of the transgenic synthetic biology pathway may be controlled by an inducible regulatory element.

Where more than one enzyme is required to form the transgenic synthetic biology pathway, expression of a rate-limiting enzyme in the transgenic synthetic biology pathway may be controlled by an inducible regulatory element.

For example, expression of the transgenic synthetic biology pathway may be induced by the binding of an antigen to the CAR or TCR; by factors present in the tumour microenvironment; or by the binding of a second small molecule to the cell.

An advantage of such control mechanisms is that the engineered cell of the present invention may express a transgenic synthetic biology pathway which produces a therapeutic small molecule which is toxic when delivered systemically.

Examples of mechanisms by which the transgenic synthetic biology pathway may be expressed in an inducible manner include, but are not limited to, (a) expression triggered by a factor in the tumour microenvironment (e.g. binding of cognate antigen to the CAR or transgenic TCR); and (b) expression trigger by a small molecule pharmaceutical.

Expression of the transgenic synthetic biology pathway which is induced by a factor in the tumour microenvironment means that the present engineered T-cell will only express the transgenic synthetic biology pathway—and thus produce the therapeutic small molecule—when it is localised to the tumour. This inducible expression is therefore expected to reduce systemic effects (e.g. toxic effects).

Illustrative mechanisms by which the expression of the transgenic synthetic biology pathway may be induced include the use of a promoter that is activated following activation of the T-cell; and the use of a scFV-Notch chimeric receptor in combination with a Notch response element to regulate expression of the transgenic synthetic biology pathway

Suitably, expression of the transgenic synthetic biology pathway (or a rate-limiting enzyme in the transgenic synthetic biology pathway) may be under the control of a promoter that is activated following activation of the T-cell. Herein, when the CAR or TCR recognizes antigen, the T-cell gets activated, transcription from the inducible promoter is stimulated and the transgenic synthetic biology pathway is provided to produce the therapeutic small molecule.

Illustrative methods to achieve induced expression following T cell activation include the use of an NFAT recognition sequence as a promotor element for the transgenic synthetic biology pathway (or a rate-limiting enzyme in the transgenic synthetic biology pathway). A consensus NFAT recognition sequence is GGAAAA (SEQ ID NO: 14). This approach has previously been used by Chmielewski et al. to achieve NFAT-dependent IL12 secretion (see Cancer Res. 71, 5697-5706 (2011)—incorporated herein by reference).

Further approaches include the use of a chimeric Notch receptor. This is a receptor which grafts a scFv onto Notch. When the scFv recognizes its cognate target, the endodomain of the receptor (which is a transcription factor) is released from the membrane and activate gene(s) in the nucleus (see Lim et al.; Cell 164, 780-791 (2016)—herein incorporated by reference).

Expression of the transgenic synthetic biology pathway (or a rate-limiting enzyme in the transgenic synthetic biology pathway) may also be induced by using a regulatory element which is activated downstream of factors which are associated with the tumour microenvironment.

Suitably, the factor is a soluble factor which is increased in a tumour microenvironment compared to a non-tumour microenvironment. For example, a factor which is increased in a tumour microenvironment may be present at a 10, 20, 50, 100, 500 or 1000-fold greater level in a tumour microenvironment compared to a non-tumour microenvironment. For example, the factor associated with a tumour microenvironment may be lactate, ornithine, adenosine, inosine, glutamate or kynurenic acid.

Approaches for detecting a soluble factor in a tumour microenvironment are described in WO 2017/029511, for example.

Expression of the transgenic synthetic biology pathway (or a rate-limiting enzyme in the transgenic synthetic biology pathway) which is induced by a small molecule pharmaceutical means that the present engineered cell will only express the transgenic synthetic biology pathway—and thus produce the therapeutic small molecule—when the small molecule pharmaceutical is administered and recognised by the cell. This inducible expression is therefore expected to reduce systemic effects (e.g. toxic effects) as the engineered cells can be induced to express the transgenic synthetic biology pathway at a time when they have localised to the tumour. In particular, expression of the transgenic synthetic biology pathway will by induced by administration of the small molecule pharmaceutical to a subject. Further, if toxicity occurs, production of the therapeutic small molecule by the transgenic synthetic biology pathway can be controlled by reducing the amount of the small molecule pharmaceutical administered or withdrawal of the small molecule pharmaceutical.

Suitable small molecule pharmaceuticals are not particularly limited and are well-known in the art. By way of example, the small molecule pharmaceutical may be selected from the following list: tetracycline, minocycline, tamoxifen, rapamycin and rapamycin analogues, the chemical inducer of dimerization AP1903 (Proc. Natl. Acad. Sci. U.S.A. 95, 10437-10442 (1998)), coumermycin, ecdysteroids and semi-synthetic ecdysteroids (Lapenna et al, ChemMedChem 4, 55-68 (2009)) and SHLD1 (Banaszynski et al, Cell 126, 995-1004 (2006)).

Expression of the transgenic synthetic biology pathway (or a rate-limiting enzyme in the transgenic synthetic biology pathway) may be achieved using a “Tet operon”. Here a protein (tetR) undergoes a conformational change which modulates its binding to a tet response DNA element in response to tetracycline. Tet transcriptional systems which switch on (Tet-on) or switch off (Tet-off) have been described and are known in the art (see Sakemura et al; Cancer Immunol. 4, 658-668 (2016)—incorporated herein by reference).

Other transcriptional switches have been described which may have advantages over the Tet system in that they are less immunogenic. Once such system is semi-synthetic O-alkyl ecdysteroid system (Rheoswitch) (see Lapenna, S. et al; ChemMedChem 4, 55-68 (2009)—incorporated herein by reference).

Further approaches to control expression of the transgenic synthetic biology pathway (or a rate-limiting enzyme in the transgenic synthetic biology pathway) with a small molecule pharmaceutical include small molecule re-complementation. Here, an enzyme is separated into two parts which do not function individually. Each part is attached to one part of a small molecule heterodimerization system (e.g. FRB/FKBP12 and rapamycin). In the presence of the drug, the enzyme is brought together, and synthesis activated. An illustrative example of this is provided by Azad et al. (Anal. Bioanal. Chem. 406, 5541-5560 (2014)—incorporated herein by reference).

A further approach to control expression of the transgenic synthetic biology pathway (or a rate-limiting enzyme in the transgenic synthetic biology pathway) with a small molecule pharmaceutical is with de-stabilizing domains. Here, certain protein domains are engineered to be unstable in the absence of a small molecule pharmaceutical. If this destabilizing domain is fused with a critical enzyme in a transgenic synthetic biology pathway, it is targeted for ubiquitination and degradation and thus synthesis of the therapeutic small molecule will be prevented. In the presence of the small molecule pharmaceutical, the destabilizing domain is stabilized and the fused enzyme does not become ubiquitinated. The transgenic synthetic biology pathway is thus able to function and produce the therapeutic small molecule. An example of this system is described by Banaszynski et al. (see Cell 126, 995-1004 (2006) & Nat. Med. 14, 1123-1127 (2008)—herein incorporated by reference).

Chimeric Antigen Receptor (CAR)

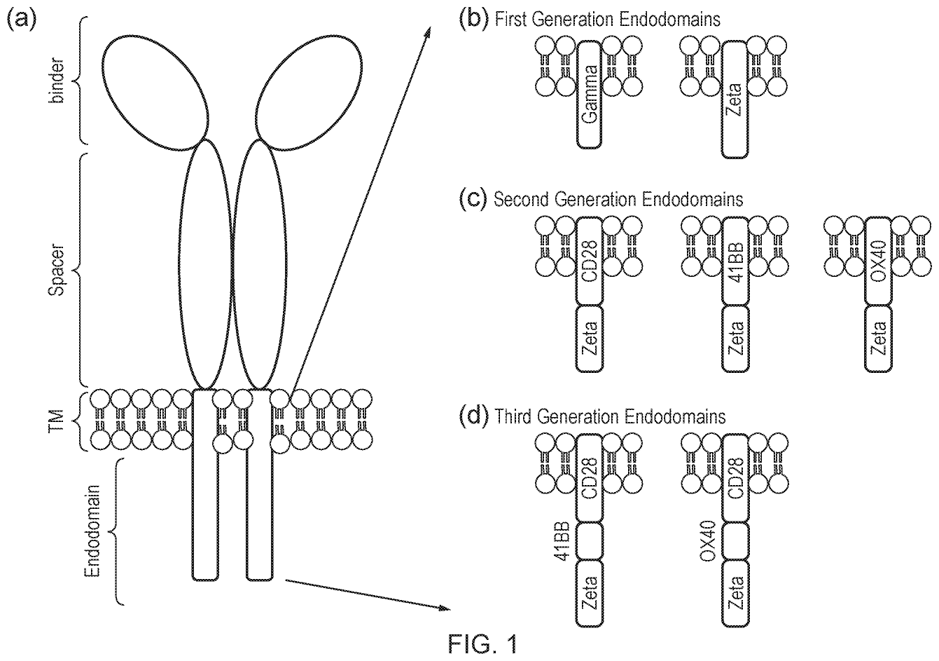

Classical CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site or on a ligand for the target antigen. A spacer domain may be necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on. Thus the CAR directs the specificity and cytotoxicity of the T cell towards cells expressing the targeted antigen.

Antigen Binding Domain

The antigen-binding domain is the portion of a classical CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor.

Various tumour associated antigens (TAA) are known, as shown in the following Table 1. The antigen-binding domain used in the present invention may be a domain which is capable of binding a TAA as indicated therein.

TABLE 1

| Cancer type | TAA |
|---|---|
| Diffuse Large B-cell Lymphoma | CD19, CD20 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

Transmembrane Domain

The transmembrane domain is the sequence of a classical CAR that spans the membrane. It may comprise a hydrophobic alpha helix. The transmembrane domain may be derived from CD28, which gives good receptor stability.

Signal Peptide

The CAR may comprise a signal peptide so that when it is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

Spacer Domain

The CAR may comprise a spacer sequence to connect the antigen-binding domain with the transmembrane domain. A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

Intracellular Signalling Domain

The intracellular signalling domain is the signal-transmission portion of a classical CAR.

The most commonly used signalling domain component is that of CD3-zeta endodomain, which contains 3 ITAMs. This transmits an activation signal to the T cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together (illustrated in FIG. 1B).

The intracellular signalling domain may be or comprise a T cell signalling domain.

The intracellular signalling domain may comprise one or more immunoreceptor tyrosine-based activation motifs (ITAMs). An ITAM is a conserved sequence of four amino acids that is repeated twice in the cytoplasmic tails of certain cell surface proteins of the immune system. The motif contains a tyrosine separated from a leucine or isoleucine by any two other amino adds, giving the signature YxxL/I. Two of these signatures are typically separated by between 6 and 8 amino adds in the tail of the molecule (YxxL/Ix₍₆₋₈₎Yxx/I).

ITAMs are important for signal transduction in immune cells. Hence, they are found in the tails of important cell signalling molecules such as the CD3 and ζ-chains of the T cell receptor complex, the CD79 alpha and beta chains of the B cell receptor complex, and certain Fc receptors. The tyrosine residues within these motifs become phosphorylated following interaction of the receptor molecules with their ligands and form docking sites for other proteins involved in the signalling pathways of the cell.

The intracellular signalling domain component may comprises, consist essentially of, or consist of the CD3-ζ endodomain, which contains three ITAMs. Classically, the CD3-endodomain transmits an activation signal to the T cell after antigen is bound. However, in the context of the present invention, the CD3-ζ endodomain transmits an activation signal to the T cell after the MHC/peptide complex comprising the engineered B2M binds to a TCR on a different T cell.

The intracellular signalling domain may comprise additional co-stimulatory signalling. For example, 4-1BB (also known as CD137) can be used with CD3-, or CD28 and OX40 can be used with CD3-ζ to transmit a proliferative/survival signal.

Accordingly, intracellular signalling domain may comprise the CD3-ζ endodomain alone, the CD3-ζ endodomain in combination with one or more co-stimulatory domains selected from 4-1BB, CD28 or OX40 endodomain, and/or a combination of some or all of 4-1BB, CD28 or OX40.

The endodomain may comprise one or more of the following: an ICOS endodomain, a CD2 endodomain, a CD27 endodomain, or a CD40 endodomain.

The endomain may comprise the sequence shown as SEQ ID NO: 15 to 18 or a variant thereof having at least 80, 85, 90, 95, 98 or 99% sequence identity, provided that the variant sequence retains the capacity to transmit an activating signal to the cell.

The percentage identity between two polypeptide sequences may be readily determined by programs such as BLAST, which is freely available at http://blast.ncbi.nlm.nih.gov. Suitably, the percentage identity is determined across the entirety of the reference and/or the query sequence.

SEQ ID NO: 15 - CD3-ζ endodomain
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR
SEQ ID NO: 16 - 4-1BB and CD3-ζ endodomains
MGNSCYNIVATLLLVLNFERTRSLQDPCSNCPAGTFCDNNRNQICSPCP
PNSFSSAGGQRTCDICRQCKGVFRTRKECSSTSNAECDCTPGFHCLGAG
CSMCEQDCKQGQELTKKGCKDCCFGTFNDQKRGICRPWTNCSLDGKSVL
VNGTKERDVVCGPSPADLSPGASSVTPPAPAREPGHSPQIISFFLALTS
TALLFLLFFLTLRFSVVKRGRKKLLYIFKQPFMRPVQTTQEEDGCSCRF
PEEEEGGCELRVKFSRSADAPAYQQGQNQLYNELNLGRREEYDVLDKRR
GRDPEMGGKPQRRKNPQEGLYNELQKDKMAEAYSEIGMKGERRRGKGHD
GLYQGLSTATKDTYDALHMQALPPR
SEQ ID NO: 17 - CD28 and CD3-ζ endodomains
SKRSRLLHSDYMNMTPRRPGPTRKHYQPYAPPRDFAAYRSRVKFSRSAD
APAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQ
ALPPR
SEQ ID NO: 18 - CD28, OX40 and CD3-ζ endodomains
SKRSRLLHSDYMNMTPRRPGPTRKHYQPYAPPRDFAAYRSRDQRLPPDA
HKPPGGGSFRTPIQEEQADAHSTLAKIRVKFSRSADAPAYQQGQNQLYN
ELNLGRREEYDVLDKRRGRDPEMGGKPRRKNPQEGLYNELQKDKMAEAY
SEIGMKGERRRGKGHDGLYQGLSTATKDTYDALHMQALPPR

Transgenic T-Cell Receptor (TCR)

The T-cell receptor (TCR) is a molecule found on the surface of T cells which is responsible for recognizing fragments of antigen as peptides bound to major histocompatibility complex (MHC) molecules.

The TCR is a heterodimer composed of two different protein chains. In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using vector. For example the genes for engineered TCRs may be reintroduced into autologous T cells and transferred back into patients for T cell adoptive therapies. Such teterologous' TCRs may also be referred to herein as ‘transgenic TCRs’.

Cell

The cell of the present invention may be an immune effector cell, such as a T-cell, a natural killer (NK) cell or a cytokine induced killer cell.

The T cell may be an alpha-beta T cell or a gamma-delta T cell.

The cell may be derived from a patient's own peripheral blood (1st party), or in the setting of a haematopoietic stem cell transplant from donor peripheral blood (2nd party), or peripheral blood from an unconnected donor (3rd party). T or NK cells, for example, may be activated and/or expanded prior to being transduced with nucleic acid molecule(s) encoding the polypeptides of the invention, for example by treatment with an anti-CD3ζ monoclonal antibody.

Alternatively, the cell may be derived from ex vivo differentiation of inducible progenitor cells or embryonic progenitor cells to T cells. Alternatively, an immortalized T-cell line which retains its lytic function may be used.

The cell may be a haematopoietic stem cell (HSC). HSCs can be obtained for transplant from the bone marrow of a suitably matched donor, by leukopheresis of peripheral blood after mobilization by administration of pharmacological doses of cytokines such as G-CSF [peripheral blood stem cells (PBSCs)], or from the umbilical cord blood (UCB) collected from the placenta after delivery. The marrow, PBSCs, or UCB may be transplanted without processing, or the HSCs may be enriched by immune selection with a monoclonal antibody to the CD34 surface antigen.

The cell of the present invention is an engineered cell. Accordingly, the first nucleic sequence encoding a CAR or transgenic TCR and one or more nucleic acid sequences which encodes one or more enzymes capable of synthesising a therapeutic small molecule are not naturally expressed by the alpha-beta T cell, a NK cell, a gamma-delta T cell or a cytokine-induced killer cell.

Nucleic Acid Construct/Kit of Nucleic Acid Sequences

The present invention provides a nucleic acid sequence which comprises: (i) a first nucleic acid sequence which encodes a chimeric antigen receptor (CAR) or a transgenic TCR; and (ii) one or more nucleic acid sequences which encode one or more enzymes which are capable of synthesising a therapeutic small molecule when expressed in combination in a cell as defined herein.

Suitably, the one or more enzymes which are capable of synthesising a therapeutic small molecule when expressed in combination in a cell are encoded on a single nucleic acid sequence.

The present invention further provides a kit comprising nucleic acid sequences according to the present invention. For example, the kit may comprise i) a first nucleic acid sequence which encodes a chimeric antigen receptor (CAR) or a transgenic TCR; and (ii) one or more nucleic acid sequences which encode one or more enzymes which are capable of synthesising a therapeutic small molecule when expressed in combination in a cell as defined herein.

Suitably, the one or more enzymes which are capable of synthesising a therapeutic small molecule when expressed in combination in a cell are encoded on a single nucleic acid sequence.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described herein to reflect the codon usage of any particular host organism in which the polypeptides are to be expressed. Suitably, the polynucleotides of the present invention are codon optimised to enable expression in a mammalian cell, in particular an immune effector cell as described herein.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

Co-Expression Site

A co-expression site is used herein to refer to a nucleic acid sequence enabling co-expression of both (i) a CAR or a TCR; and (ii) one or more enzymes which are capable of synthesising a therapeutic small molecule when expressed in combination in a cell as defined herein.

The co-expression site may be a sequence encoding a cleavage site, such that the nucleic acid construct produces comprises the two polypeptides joined by a cleavage site(s). The cleavage site may be self-cleaving, such that when the polypeptide is produced, it is immediately cleaved into individual peptides without the need for any external cleavage activity. Suitable self-cleaving polypeptides are described herein.

The co-expressing sequence may be an internal ribosome entry sequence (IRES). The co-expressing sequence may be an internal promoter.

Vector

The present invention also provides a vector, or kit of vectors which comprises one or more nucleic acid sequence(s) or nucleic acid construct(s) of the invention. Such a vector may be used to introduce the nucleic acid sequence(s) or construct(s) into a host cell so that it expresses a CAR or transgenic TCR and one or more enzymes which are capable of synthesising a therapeutic small molecule when expressed in combination in the cell.

The vector may, for example, be a plasmid or a viral vector, such as a retroviral vector or a lentiviral vector, or a transposon based vector or synthetic mRNA.

The vector may be capable of transfecting or transducing a cell.

Pharmaceutical Composition

The present invention also relates to a pharmaceutical composition containing a cell, a nucleic acid construct, a first nucleic acid sequence and a second nucleic acid sequence; a vector or a first and a second vector of the present invention. In particular, the invention relates to a pharmaceutical composition containing a cell according to the present invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The present invention provides a method for treating and/or preventing a disease which comprises the step of administering the cells of the present invention (for example in a pharmaceutical composition as described above) to a subject.

A method for treating a disease relates to the therapeutic use of the cells of the present invention. In this respect, the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:

(i) isolating a cell-containing sample;

(ii) transducing or transfecting such cells with a nucleic acid sequence or vector provided by the present invention;

(iii) administering the cells from (ii) to a subject.

The present invention provides a cell, a nucleic acid construct, a first nucleic acid sequence and a second nucleic acid sequence, a vector, or a first and a second vector of the present invention for use in treating and/or preventing a disease. In particular the present invention provides a cell of the present invention for use in treating and/or preventing a disease

The invention also relates to the use of a cell, a nucleic acid construct, a first nucleic acid sequence and a second nucleic acid sequence, a vector, or a first and a second vector of the present invention of the present invention in the manufacture of a medicament for the treatment and/or prevention of a disease. In particular, the invention relates to the use of a cell in the manufacture of a medicament for the treatment and/or prevention of a disease

The disease to be treated and/or prevented by the method of the present invention may be immune rejection of the cell which comprises (i) a chimeric antigen receptor (CAR) or a transgenic TCR; and (ii) one or more enzymes which are capable of synthesising a therapeutic small molecule when expressed in combination in a cell as defined herein.

The methods may be for the treatment of a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

Preferably, the methods may be for the treatment of a solid tumour, such as bladder cancer, breast cancer, colon cancer, endometrial cancer, kidney cancer (renal cell), lung cancer, melanoma, neuroblastoma, sarcoma, glioma, pancreatic cancer, prostate cancer and thyroid cancer.

The cell, in particular the CAR cell, of the present invention may be capable of killing target cells, such as cancer cells. The target cell may be recognisable by expression of a TAA, for example the expression of a TAA provided above in Table 1.

Method of Making a Cell

CAR or transgenic TCR-expressing cells of the present invention may be generated by introducing DNA or RNA coding for the CAR or TCR and one or more enzymes which are capable of synthesising a therapeutic small molecule when expressed in combination in the cell by one of many means including transduction with a viral vector, transfection with DNA or RNA.

The cell of the invention may be made by:

(i) isolation of a cell-containing sample from a subject or one of the other sources listed above; and

(ii) transduction or transfection of the cells with one or more a nucleic acid sequence(s) or nucleic acid construct as defined above in vitro or ex vivo.

The cells may then by purified, for example, selected on the basis of expression of the antigen-binding domain of the antigen-binding polypeptide.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1—Violacein Production in Mammalian Cells

Violacein is a tryptophan derivative synthesized by a number of bacterial species. It is made by a complex biosynthetic pathway which also generates the recognised anticancer drugs rebeccamycin and staurosporine (FIG. 2a).

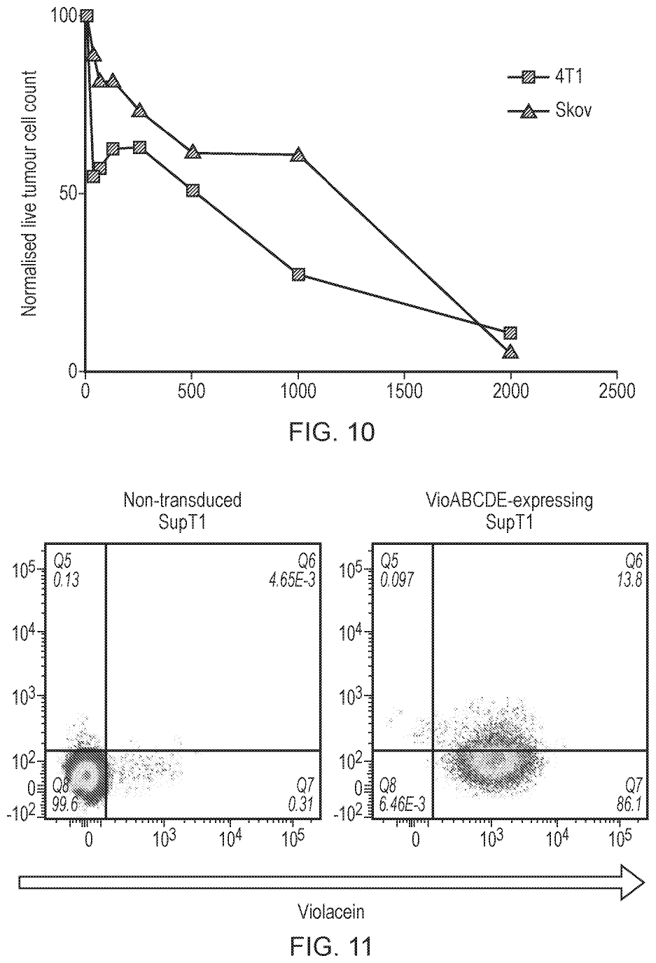

Initial studies showed were carried out to measure the sensitivity of two tumour cell lines (4T1 and Skov) to violacein as follows: adherent cells were plated at a density of 2×10⁴/well in a 24-well plate and allowed to adhere for 24 hours. Cells were then incubated with the indicated concentration of violacein for 72 hours. Cells were harvested and live cells enumerated and normalized to vehicle-treated control (which was set to 100%). The results are shown in FIG. 10.

Synthesis of violacein requires a biosynthetic operon consisting of 5 genes VioA, B, C, D and E (FIG. 2b). This operon was split into 2 separate retroviral expression plasmids containing the VioA and VioB genes, and the VioC, VioD and VioE genes respectively. Expression of all 5 genes are required for violacein production.

The violacein biosynthetic genes were introduced into SupT1 cells by retroviral transduction. Due to the natural fluorescence of violacein, it was possible to measure violacein production in SupT1 T cell line using flow cytometry analysis (FIG. 11).

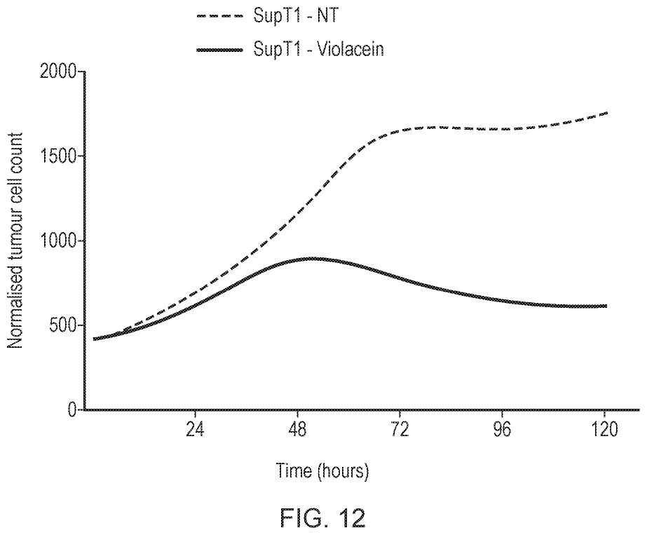

Incubation of violacein-producing SupT1 T cells with SKOV3 cells demonstrated that violacein production resulted in a suppression of SKOV3 cell growth (FIG. 12). In order to demonstrate the sensitivity of the SKOV3 cells to violacein, SupT1 expressing the Violacein biosynthetic operon and thus synthesising violacein were co-cultured with SKOV3 cells as follows: SKOV3 cells expressing a nuclear-localized red fluorescent protein (mKATE) were plated in a 96-well plate at a density of 10,000 cells per well and allowed to adhere overnight. The following day the indicated supT1 cells were added to the SKOV3 cells at density of 20,000 cells per well in a total volume of 200 ul cell culture medium. Cells were continuously monitored in a Incycute live cell imager and the number of viable SKOV3 cells enumerated every hour by counting the presence of red fluorescent nuclei.

### Example 2—Effect of Violacein on CAR T-Cell Function in AML

Normal human T-cells are transduced with a CAR which recognizes the myeloid antigen CD33 along with the lentiviral vector described above which codes for Violacein. Control T-cells are also generated which are only transduced with the CD33 CAR. Non-transduced T-cells from the same donor, CD33 CAR T-cells and CD33 CAR/Violecein T-cells are co-cultured with the AML cell line HL60 at different effector to target ratios for 1, 2, 5 and 7 days. Quantity of remaining HL60 target cells is determined by flow cytometry. An NSG mouse model of AML using HL60 cells is tested by treating with CD33 CAR cells and CD33 CAR/Violacein cells.

### Example 3—Geraniol Production

Geraniol is a monoterpenoid compound synthesized by many plant species which displays an anti-proliferative/pro-apoptotic effect against cancer cells in vitro. It is produced from the precursor geranyl diphosphate by the action of the enzyme geraniol synthase. Additionally, geranyl diphosphate is a product of the mevalonate pathway in human cells which lack geraniol synthase.

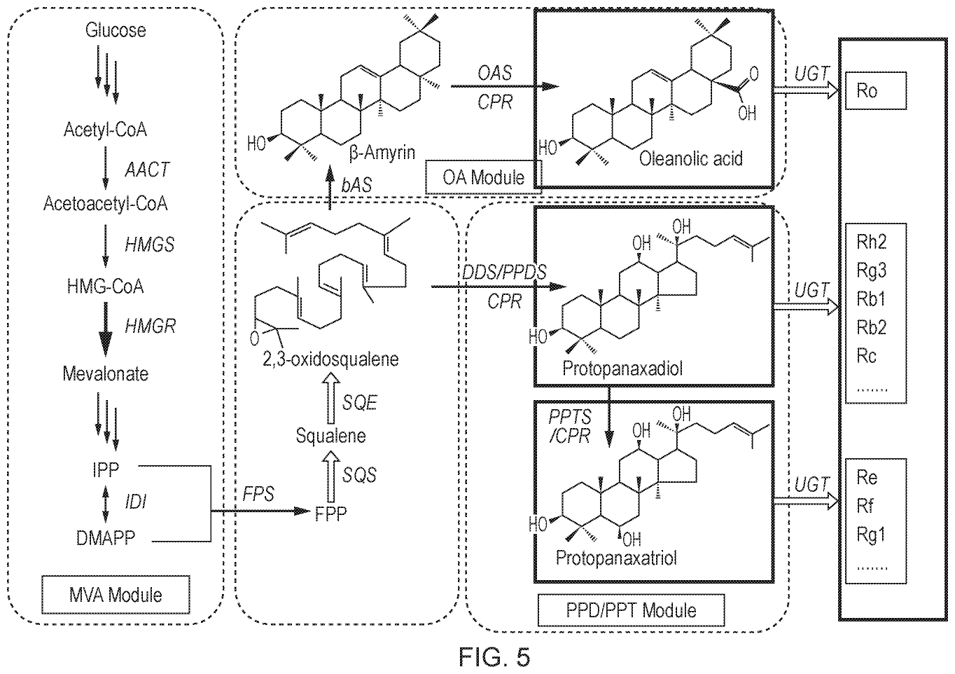

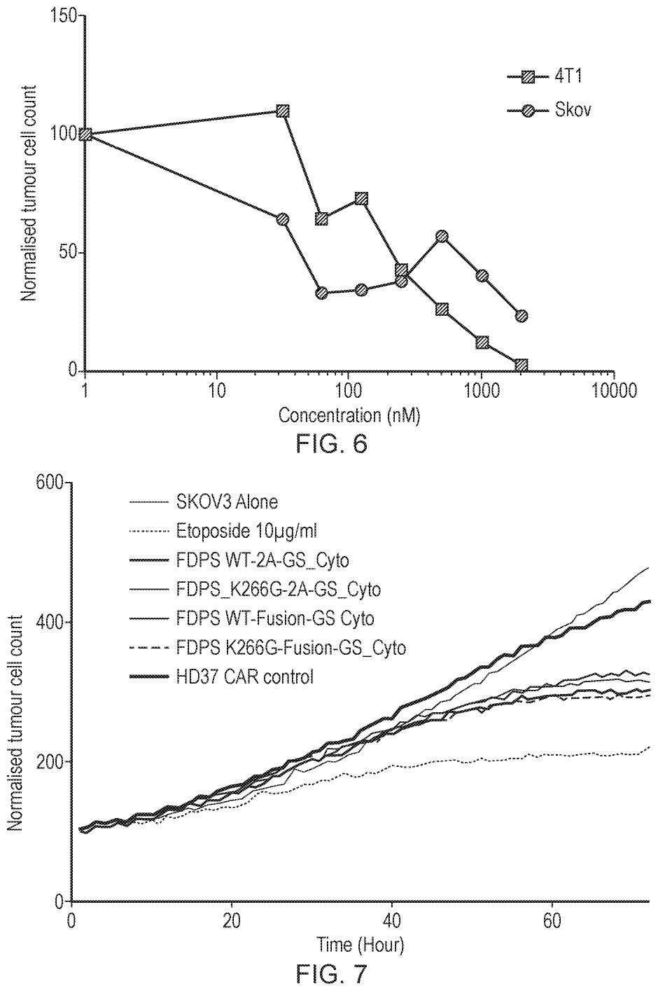

In order to test the sensitivity of tumour cell lines to geraniol, SKOV3 ovarian cancer cells or 4T1 breast cancer cells were plated out at a density of 2×10⁴ cells per well in a 48-well plate and incubated for the 24 hours with the indicated concentration of geraniol (FIG. 6). Cells were then harvested and viable cells enumerated and normalized to the number of live cells in vehicle-wells (which is set to 100%).

Production of geraniol in the SupT1 T cell line was initiated by introduction through retroviral transduction of the geraniol synthase (GS) gene from Valeria officinalis co-expressed with the human farnesyl diphosphate synthase (FDPS) gene, either as a separate enzyme or fused directly to geraniol synthase, which was introduced to boost production of precursor geranyl diphosphate molecules from the host cell metabolic pathway (see table below). All constructs were co-expressed with an anti-CD19 CAR based upon the anti-CD19 antibody HD37 and possessing a 41BB and CD3zeta endodomain. In some cases, the FDPS also contained the K266G mutation which has been reported to enhance geraniol phosphate production.

| Construct | Description |
|---|---|
| FDPS WT-2A-GS_Cyto | Wild type FDPS co-expressed |
| | separately with geraniol synthase |
| FDPS_K266G -2A-GS_Cyto | K226G-mutated FDPS co-expressed |
| | with geraniol synthase |
| FDPS WT-Fusion-GS Cyto | Wild type FDPS co-expressed fused |
| | directly to geraniol synthase |
| FDPS_K266G-Fusion-GS_Cyto | K266G-mutated FDPS co-expressed |
| | fused directly to geraniol synthase |

In order to demonstrate the sensitivity of the ovarian SKOV3 cell line to geraniol, SupT1 expressing the FDPS and GS constructs listed in the above table were co-cultured with SKOV3 cells as follows: SKOV3 cells expressing a nuclear-localized red fluorescent protein (mKATE) were plated in a 96-well plate at a density of 5,000 cells per well and allowed to adhere overnight. The following day the indicated transduced SupT1 cells were added to the SKOV3 cells at density of 20,000 cells per well in a total volume of 200 ul cell culture medium. Etoposide, which induces the apoptosis of SKOV3 cells, was used a positive control of cell killing/inhibition at a concentration of 10 ug/ml. Cells were continuously monitored in a Incycute live cell imager and the number of viable SKOV3 cells enumerated every hour by counting the presence of red fluorescent nuclei.

Co-culture of SupT1 T cells expressing these constructs with CD19-negative SKOV3 ovarian cancer cell line resulted in increased growth inhibition of SKOV3 cells when compared to the control CAR lacking the geraniol producing GS gene (FIG. 7).

### Example 4—Caffeine Production

Caffeine is a purine derivative synthesized by a number of plant species and is a known antagonist of the immunomodulatory Adenosine A2AR receptor expressed on T cells.

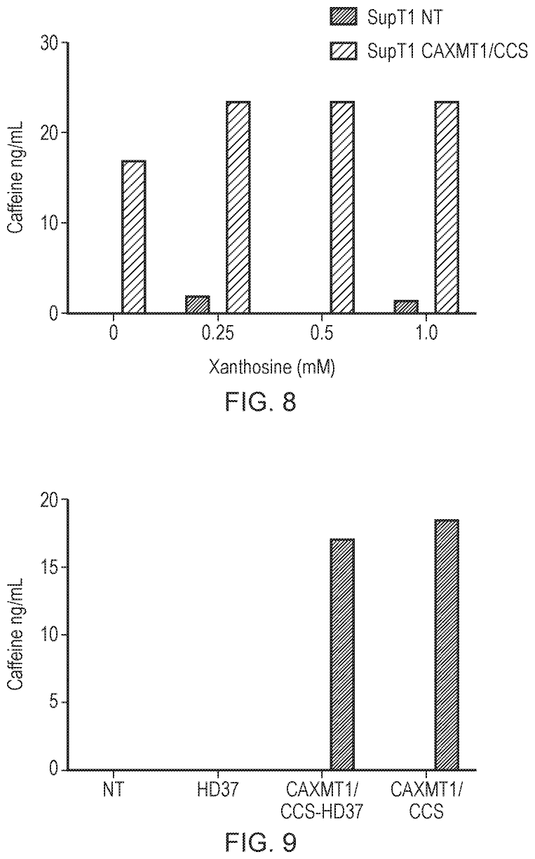

Introduction of the caffeine biosynthetic genes Caffeine methyl transferase (CAXMT1) from Coffea arabica and caffeine synthase (CCS1) from Camellia sinensis into the SupT1 T cell line resulted in the production of caffeine by these human cell lines. Caffeine production could be further enhanced by the addition of the pre-cursor xanthosine (FIG. 8). The production of caffeine was monitored by culturing 1×10⁶ transduced cells in a 2 ml culture medium in the presence of the indicated amounts of Xanthosine. After 72 hours supernatants were harvested, cleared of cells by centrifugation and caffeine levels were determined by ELISA.

The production of caffeine was also observed in human primary PBMCs retrovirally transduced with the CAXMT1 and CCS genes with and without a CD19 CAR (HD37) (FIG. 9). The production of caffeine was monitored by culturing 5×10⁵ transduced cells in the presence of the 50 uM xanthosine. After 72 hours supernatants were harvested, cleared of cells by centrifugation and caffeine levels determined by ELISA.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. An engineered cell which comprises;
(i) a chimeric antigen receptor (CAR) or a transgenic T-cell receptor (TCR); and
(ii) one or more engineered polynucleotides which encode one or more enzymes which are capable of synthesising a therapeutic small molecule when expressed in combination in the cell.

2. A cell according to claim 1 wherein the one or more engineered polynucleotides encode at least two enzymes.

3. A cell according to claim 2 wherein the at least two enzymes are encoded by one engineered polynucleotide.

4. A cell according to claim 3 wherein the engineered polynucleotide is an operon.

5. A cell according to claim 2 wherein the at least two enzymes are encoded in a single open reading frame and each enzyme is separated by a cleavage site.

6. (canceled)

7. A cell according to claim 1 wherein the therapeutic small molecule is selected from a cytotoxic molecule; a cytostatic molecule; an agent which is capable of inducing differentiation of the tumour; and a proinflammatory molecule.

8.-10. (canceled)

11. A cell according to claim 1 wherein the engineered cell is further engineered to have reduced sensitivity to the therapeutic small molecule.

12. (canceled)

13. A cell according to claim 1 wherein expression of the one or more of enzymes is induced by the binding of an antigen to the CAR or transgenic TCR.

14. A cell according to claim 1 wherein expression of the one or more of enzymes is induced by a tumour microenvironment.

15. A cell according to claim 1 wherein expression of the one or more of enzymes is induced by the binding of a second small molecule to the cell.

16. (canceled)

17. A nucleic acid construct which comprises:
(i) a first nucleic acid sequence which encodes a chimeric antigen receptor (CAR) or a transgenic TCR; and
(ii) one or more nucleic acid sequences which encode one or more enzymes which are capable of synthesising a therapeutic small molecule when expressed in combination in a cell.

18. (canceled)

19. A kit of nucleic acid sequences comprising:
(i) a first nucleic acid sequence which encodes a chimeric antigen receptor (CAR) or a transgenic TCR; and
(ii) one or more nucleic acid sequences which encode one or more enzymes which are capable of synthesising a therapeutic small molecule when expressed in combination in a cell.

20. A vector which comprises a nucleic acid construct according to claim 17.

21. A kit of vectors which comprises:
(i) a first vector which comprises a nucleic acid sequence which encodes a chimeric antigen receptor (CAR) or a transgenic TCR; and
(ii) a second vector which comprises one or more enzymes which are capable of synthesising a therapeutic small molecule when expressed in combination in a cell.

22. (canceled)

23. A pharmaceutical composition which comprises a plurality of cells according to claim 1.

24. (canceled)

25. A method for treating cancer, which comprises the step of administering a pharmaceutical composition according to claim 23 to a subject in need thereof.

26. A method according to claim 25, which comprise the following steps:
(i) isolation of a cell containing sample;
(ii) transduction or transfection of the cell with: a nucleic acid sequence which encodes a chimeric antigen receptor (CAR) or a transgenic TCR; and one or more nucleic acid sequences which encode one or more enzymes which are capable of synthesizing a therapeutic small molecule when expressed in combination in a cell; and
(iii) administering the cells from (ii) to a subject.

27.-30. (canceled)

31. A method according to claim 25, wherein the cancer is a solid tumour cancer.

32. A method for making a cell according to claim 1 which comprises the step of introducing:
(i) a first nucleic acid sequence which encodes a chimeric antigen receptor (CAR) or a transgenic TCR; and
(ii) one or more nucleic acid sequences which encode one or more enzymes which are capable of synthesising a therapeutic small molecule when expressed in combination, into a cell.

33. (canceled)
